(19) United States
(12) Patent Application Publication
(10) Pub. No.: US 2025/0064740 A1
(43) Pub. Date: Feb. 27, 2025

(54) MULTI-VESICULAR LIPID NANOPARTICLE TOLEROGENIC VACCINES FOR INDUCTION OF SYSTEMIC IMMUNE TOLERANCE IN VIVO

(71) Applicant: KARMA BIOTECHNOLOGIES, Torrance, CA (US)

(72) Inventors: Andrew Brovko GRAY, Los Angeles, CA (US); Alan Michael JOHNSON, Monrovia, CA (US); Xazmin Helen CUSTER, Torrance, CA (US); Mariana FIGUEIREDO UCHOA, Torrance, CA (US); Luciana Cecilia VEIRAS, Torrance, CA (US)

(73) Assignee: KARMA BIOTECHNOLOGIES, Torrance, CA (US)

(21) Appl. No.: 18/728,701

(22) PCT Filed: Jan. 17, 2023
(86) PCT No.: PCT/US2023/010952

§ 371 Date: Jul. 12, 2024

Related U.S. Application Data
(60) Provisional application No. 63/299,669, filed on Jan. 14, 2022.

(51) Int. Cl. A61K 9/127; A61K 9/51; A61K 45/06; B82Y 5/00

(52) U.S. Cl. CPC A61K 9/1271; A61K 9/1277; A61K 9/5123; A61K 9/5192; A61K 45/06; B82Y 5/00

(57) ABSTRACT

A multivesicular lipid nanoparticle composition includes a plurality of multivesicular lipids. Each multivesicular lipid nanoparticle includes a carrier phospholipid micelle including a carrier phospholipid layer and having an average diameter less than 1 micron; and at least one sub-chamber phospholipid inverse micelle including a sub-chamber phospholipid layer and having an average diameter less than 100 nm. The carrier micelle nanoparticle encapsulates the at least one sub-chamber inverse micelle nanoparticle. Characteristically, phospholipid tail groups of the carrier micelle are covalently linked to the phospholipid tail groups of at least one sub-chamber inverse micelle nanoparticle.

### CROSS-REFERENCE TO RELATED APPLICATIONS

This application claims the benefit of U.S. provisional application Ser. No. 63/299,669 filed Jan. 14, 2022, the disclosure of which is hereby incorporated in its entirety by reference herein.

### TECHNICAL FIELD

The invention generally relates to controlled encapsulation and delivery of biomolecules that can be characterized as small molecule drugs, nucleotides, and biologics to immune cells to induce a state of immune tolerance in vivo and in vitro. In particular, this invention relates to improvements made in delivery, uptake and stimuli-responsive expression of genes for cellular reprogramming of immune cells to treat and prevent autoimmune diseases, allergies, and food sensitivities.

### BACKGROUND

Vertebrates have evolved complex immune systems to protect them from pathogens, parasites, and harmful environmental substances. Under normal circumstances, these immune systems can differentiate between these threats and the cells and tissues of their own bodies (and other harmless substances). The cells and tissues that the immune system determines to be “self” are said to be “tolerated”. This self-tolerance is initiated during early development of the organism (central tolerance) and is maintained throughout life (peripheral tolerance) by a wide variety of cellular and molecular mechanisms such as by the actions of regulatory T cells (Tregs). When these mechanisms fail and a component of a cell or tissue is no longer recognized as “self”, it becomes an autoantigen and autoimmunity and autoimmune disease can result. Similarly, peripheral tolerance mechanisms can train the immune system to recognize common environmental substances (such as house dust and pollen) as harmless, and therefore they are tolerated by the immune system. When this process fails, allergies and food sensitivities result. Tolerance to common allergens has been restored in some cases using crude methodologies such as “allergy shots”, where the patient's immune system is repeatedly stimulated with small amounts of the allergen in question. Recently this approach has been formalized and has been successful, for example during efforts to minimize peanut allergies in children by repeatedly feeding them minute quantities of peanuts. However, this approach is not feasible for many allergens, is not targeted in any way, and is not capable of inducing a strong enough tolerogenic effect to treat or prevent autoimmune diseases. To achieve this, a true tolerogenic vaccine is necessary. Where a standard vaccine activates cells of the immune system and shows it an example of a pathogen that it should attack, a tolerogenic vaccine puts certain cells of the immune system into a tolerogenic state and gives them an example of autoantigen(s) that it should tolerate. The development of a broadly applicable tolerogenic vaccine technology to induce systemic antigen-specific immune tolerance to any autoantigen or allergen are the driving factors of this innovation.

Liposomal and lipid nanoparticle systems are soft material-based nanoparticles and have been in use as carriers within extracellular environments as naturally occurring extracellular vesicles (EVs), or synthetically formulated vesicles from phospholipid formulations. Naturally occurring exosomes are known to encapsulate and deliver microRNA and protein to signal other cells of relative health, or oxidative stress. Recent innovations utilize liposome-based carriers for delivery of cytotoxic drugs to disease state tissues and cells. Interest in the delivery of nucleotides and other gene editing tools, such as CRISPR/Cas9 have driven the inherent potential of liposome systems as biomimetic carriers, that will be naturally endocytosed by the healthy and/or disease state cells and tissues. Critically important improvements in encapsulation and stabilization of vesicle-packaged biomolecules for eventual delivery are the driving factors of this innovation. Current lipid nanoparticle vaccines are severely hampered by their instability at room temperature, requiring a complex and expensive logistical chain involving storage at sub-zero temperatures. Once thawed, current lipid nanoparticle vaccines must be refrigerated and used within days, or be wasted. A critical feature of this innovation is its extreme stability, the lipid nanoparticles being stable for months at high ambient temperatures rather than days in a refrigerator.

The prior art for this matter is the use of cellular and molecular methods to induce immune tolerance in vivo. Fundamentally, all of these methods are similar in that they seek to deliver a protein/peptide autoantigen (or nucleic acids encoding that autoantigen) to immune cells in a tolerogenic context. The aim of these technologies is to induce antigen presentation of autoantigens by tolerogenic immune cells such as tolerogenic dendritic cells (tolDC). The tolDC then mediate peripheral immune tolerance to those autoantigens by a variety of mechanisms, such as the induction of regulatory T cells (Tregs). Cell-free methods that have been trialed include delivery of nucleic acids encoding autoantigen(s) in vivo either directly as a plasmid DNA vaccine, or indirectly via viral vectors or nanoparticles. Alternatively, the protein structure of the autoantigen itself can be delivered in vivo directly, either as the whole autoantigen protein or a smaller fragment thereof (a peptide vaccine). These cell-free methods have the advantage of being relatively straightforward to manufacture and administer, but are lacking because they do not necessarily deliver their payloads in the correct manner to the correct immune cell type to elicit a therapeutic tolerogenic response. Furthermore, current cell-free methods can generally only deliver a single class of payload (e.g., an autoantigen protein or an mRNA encoding an autoantigen protein), a serious limitation that precludes engineering immune cells simultaneously with several different classes of payloads to optimize the tolerogenic immune response (e.g., concomitant delivery of an mRNA encoding an autoantigen plus a cytokine protein in the same nanoparticle). Cell-based methods that have been trialed include ex vivo engineering and expansion of Tregs or tolDCs and their subsequent reinfusion into the patient. These methods have the advantage of reliably producing the correct tolerogenic cell type, but each treatment is patient specific and is expensive and difficult to manufacture. Currently there is no composition of matter or method for delivering multiple payloads designed to engineer T cells or DCs into therapeutically relevant Tregs or tolDCs in vivo.

Structurally, lipid nanoparticle carriers are formed by layers of phospholipids and other lipids. Phospholipid head groups are oriented to be exposed to the aqueous environment, making them ideal for functionalizing small molecule components to improve overall cellular uptake. Phospholipids and other lipids are weakly held together in bilayers by Van der Waals interactions. As a result of the weakly associated lipids, dissipation of lipid nanoparticles before complete circulation is a major deficiency. Known as multivesicular liposomes (MVLs), naturally occurring microvesicles (MVs) are large (>1 μm) single bilayer liposomes known to encapsulate multiple smaller vesicles (200 nm), forming non-concentric chambers. These chambers contain biomolecules, such as nucleotides, small molecules, metabolites, and proteins. Synthetically formulated MVLs have been demonstrated to encapsulate and deliver anti-cancer drugs and other small molecule chemotherapies.^{1,2} Key to the prior art, release of biologically active therapeutics was controlled by the difference in salt concentration between the smaller vesicle compartments and the larger encapsulating vesicle. Other soft material-based nanoparticles are polymer, peptide, and protein-based materials.

### SUMMARY

In at least one aspect, a multivesicular lipid nanoparticle composition includes a plurality of multivesicular lipid nanoparticles. Each multivesicular lipid nanoparticle includes a carrier lipid nanoparticle that includes a carrier micelle. The carrier micelle is composed of carrier lipids selected from the group consisting of phospholipids, lipids not including phosphorus, and combinations thereof, the carrier lipid nanoparticle having an average diameter less than 1 micron. The multivesicular lipid nanoparticle composition also includes at least one sub-chamber lipid nanoparticle including a sub-chamber inverse micelle. The sub-chamber inverse micelle is composed of sub-chamber lipids selected from the group consisting of phospholipids, lipids not including phosphorus, and combinations thereof. Characteristically, at least one sub-chamber lipid nanoparticle has an average diameter less than 100 nm with the carrier lipid nanoparticle encapsulating at least one sub-chamber lipid nanoparticle.

In another aspect, a multivesicular lipid nanoparticle composition that includes a plurality of multivesicular lipid nanoparticles is provided. Each multivesicular lipid nanoparticle includes a carrier micelle lipid nanoparticle including a carrier phospholipid layer comprising carrier phospholipids and having an average diameter less than 1 micron; and at least one sub-chamber inverse micelle lipid nanoparticle including a sub-chamber phospholipid layer comprising sub-chamber phospholipids and having an average diameter equal to or less than about 100 nm. The carrier lipid nanoparticle micelle encapsulates at least one sub-chamber inverse micelle nanoparticle. Critically important to this innovation is the physical cross-linking of some of the carrier micelle phospholipid tails to some of the phospholipid tails of the sub-chamber inverse micelle(s), which greatly improves the stability and release kinetics of the present invention compared to similar technologies lacking these stabilizing crosslinks. This multivesicular lipid nanoparticle composition is herein called SNIPR (Sub-Nanoparticle Intracellular Payload Release).

In another aspect, a method for forming the multivesicular lipid nanoparticle composition set forth above is provided. The method includes a step of forming a plurality of lipid nanoparticles where the plurality of carrier micelle lipid nanoparticles have an average nanoparticle diameter less than about 1 micron and the plurality of inverse micelle sub-chamber lipid nanoparticles have an average diameter of less than 100 nanometers. The plurality of lipid nanoparticles are formed from a lipid nanoparticle-forming composition that includes one or more payloads that are encapsulated within either the carrier micelle or the sub-chamber inverse micelle(s), depending on the physicochemical properties of the payload(s). The plurality of lipid nanoparticles is then subject to a stabilizing crosslinking process that covalently attaches the tail groups of certain phospholipids in the carrier micelle lipid nanoparticle to the tail groups of certain phospholipids in the inverse micelle sub-chamber lipid nanoparticles.

In another aspect, a method of forming a multivesicular lipid composition including a plurality of multivesicular lipids is provided. Each multivesicular lipid nanoparticle includes a carrier phospholipid micelle including a carrier phospholipid layer comprising carrier phospholipids and having an average diameter less than 1 micron and at least one sub-chamber phospholipid inverse micelle including a sub-chamber phospholipid layer comprising sub-chamber phospholipids and having an average diameter less than 100 nm. The carrier phospholipid micelle encapsulates the at least one sub-chamber phospholipid inverse micelle. The method includes steps of:
a) combining an organic solvent in which carrier phospholipids, sub-chamber phospholipids, and optional hydrophobic therapeutic/diagnostic payloads are dissolved with an aqueous solvent in which a hydrophilic therapeutic/diagnostic payload is dissolved; and
b) forming a plurality of sub-chamber phospholipid inverse micelles, the plurality of sub-chamber phospholipid inverse micelles having an average nanoparticle diameter less than 100 nm, the plurality of sub-chamber phospholipid inverse micelles being formed from a first MVL-forming composition that includes a hydrophilic therapeutic/diagnostic payload; and
c) forming a plurality of carrier phospholipid micelles having an average nanoparticle diameter less than 1 micron, the plurality of carrier phospholipid micelles being formed from second MVL-forming composition that includes the plurality of sub-chamber phospholipid inverse micelles such that sub-chamber phospholipid inverse micelles are encapsulated by carrier phospholipid micelles (it should be appreciate that steps b and c can occur simultaneously, especially during the mixing process); and
d) covalently linking phospholipid tail groups of the plurality of carrier phospholipid micelles and the plurality of sub-chamber phospholipid inverse micelles via a chemical reaction catalyzed by application of actinic radiation.

In another aspect, conjugated tail group covalent linkages attach sub-chamber phospholipid inverse micelles to an inner surface of the carrier lipid phospholipid micelles.

In another aspect, a multivesicular lipid nanoparticle composition that delivers payloads designed to induce antigen-specific immune tolerance is provided. Each tolerogenic multivesicular lipid nanoparticle includes a carrier micelle lipid nanoparticle including a carrier phospholipid layer comprising carrier phospholipids and optionally other lipids not containing phosphorus. The carrier phospholipid layer has an average diameter less than 1 micron; and at least one sub-chamber inverse micelle lipid nanoparticle including a sub-chamber phospholipid bilayer comprising sub-chamber phospholipids and having an average diameter equal to or less than about 100 nm. The carrier lipid nanoparticle micelle encapsulates at least one sub-chamber inverse micelle nanoparticle. In one variation, the sub-chamber inverse micelle nanoparticles encapsulate one or more hydrophilic payloads (e.g., nucleic acids, proteins, or peptides) designed to induce presentation of autoantigen epitopes by antigen-presenting cells that take up the lipid nanoparticle composition. In another variation, the sub-chamber inverse micelle nanoparticles encapsulate one or more hydrophilic payloads (e.g., nucleic acids, proteins, or peptides) designed to induce presentation of autoantigen epitopes by antigen-presenting cells that take up the lipid nanoparticle composition and the lipid nanoparticle composition also encapsulates one or more payloads (e.g., nucleic acids, proteins, peptides, or small molecule drugs) that induce a tolerogenic phenotype in antigen-presenting cells that take up the lipid nanoparticle composition. In this variation, hydrophilic payloads that induce a tolerance phenotype are encapsulated in the sub-chamber inverse micelles, and hydrophobic payloads that induce a tolerance phenotype are encapsulated within the carrier micelle lipid nanoparticle with at least one sub-chamber inverse micelle. SNIPR nanoparticles containing payloads designed to induce antigen-specific tolerance in this manner are herein called Xavine™ nanoparticles.

### BRIEF DESCRIPTION OF THE DRAWINGS

For a further understanding of the nature, objects, and advantages of the present invention, reference should be had to the following detailed description, read in conjunction with the following drawings, wherein like reference numerals denote like elements and wherein:

FIG. 1: Schematic of a multivesicular SNIPR lipid nanoparticle.

FIG. 2: Schematic of principle apparatus components (phospholipids and other components) of SNIPR lipid nanoparticles in one embodiment.

FIGS. 3A, 3B, 3C, 3D, 3E, AND 3F: Schematic of principle apparatus and examples of payloads. Multiple different payloads can be loaded into the hydrophilic cores of the lipid nanoparticle sub-chambers and/or the hydrophobic core of the main lipid nanoparticle. These payloads include, but are not limited to, one or more small molecule drugs in sub-chambers (A), one or more polynucleotides in sub-chambers (B), antibody in the sub-chambers and one or more small molecule drugs in the main lipid nanoparticle (C), one or more small molecule drugs in the main lipid nanoparticle and one or more small molecule drugs in sub-chambers (D), different small molecule drugs in individual sub-chambers (E), and, one or more polynucleotides and one or more small molecule drugs in individual sub-chambers (F).

FIG. 4: Schematic of principle apparatus and examples of payloads for Xavine™ applications.

FIG. 5: Schematic manufacturing process for SNIPR and Xavine™ lipid nanoparticles.

FIG. 6: Schematic representation of phospholipid head and tail group interactions.

FIG. 7: Modeling of phospholipid bilayers with and without PEG. Minimum free energy as a function of curvature for a lipid/PEG-lipid bilayer containing 9.15% (solid line), 9.5% (dashed line), and 9.7% (dotted line) of PEG-lipid.⁵

FIGS. 8A and 8B: Analysis of physical characteristics of empty and DNA-loaded SNIPR nanoparticles. (A) Mean empty SNIPR nanoparticle diameter, polydispersity, and nanoparticle concentration. (B) Mean pDNA-loaded SNIPR nanoparticle diameter, polydispersity, and nanoparticle concentration.

FIGS. 9A and 9B: Visualization and quantitation of pDNA loaded into SNIPR invention after fractionation by size exclusion chromatography. (A) SYBR Safe agarose gel visualization of DNA encapsulated within SNIPR nanoparticles in fractions eluted from a size exclusion column. (B) Quantitation of pDNA encapsulated within SNIPR by quantitative real-time PCR in the same fractions eluted from the size exclusion chromatography (SEC) column.

FIGS. 10A-1, 10A-2, 10A-3, 10B-1, 10B-2, 10B-3, 10C, 10D, 10E-1, 10E-2, and 10F: Determination of mechanism of uptake of SNIPR lipid nanoparticles by cells. (A) Quantitation of uptake of fluorescently-labeled SNIPR nanoparticles by human THP-1 cells at a range of SNIPR concentrations at two time points, 30 minutes and two hours. (B) Quantitation of uptake of fluorescently-labeled SNIPR nanoparticles by the human HEK-293 cell line at 24 hours after treatment with a range of SNIPR concentrations. (C) Quantitation of uptake of fluorescently-labeled SNIPR nanoparticles by various primary immune cells (T cells, natural killer cells, monocytes, dendritic cells, and B cells) at a range of SNIPR concentrations. (D) Overview of uptake mechanisms commonly used by lipid nanoparticles to enter cells. (E) Transfection of a T cell line by pDNA-loaded SNIPR nanoparticles is abrogated by the caveolin-mediated uptake inhibitor Methyl-beta-cyclodextrin (MbC). (F) Transfection of a liver cell line by pDNA-loaded SNIPR nanoparticles is abrogated by the caveolin-mediated uptake inhibitor MbC.

FIGS. 11A, 11B, 11C, 11D, 11E, and 11F: Quantitation of transfection efficiency by plasmid-loaded SNIPR lipid nanoparticles in multiple cell types. (A) Fluorescence imaging of EGFP expression in human HEK-293 cells treated with SNIPR[DNA] nanoparticles loaded with a plasmid encoding EGFP at a range of concentrations. (B) Flow cytometric quantitation of transfection efficiency (percentage EGFP positive cells) in HEK-293 cell line cells treated with SNIPR[pDNA-EGFP]. (C) Flow cytometric quantitation of intensity of EGFP expression (Mean Fluorescence Intensity, MFI, of EGFP positive cells) in HEK-293 cell line cells treated with SNIPR[pDNA-EGFP]. (D) Flow cytometric quantitation of uptake of fluorescently-labeled SNIPR nanoparticles by mouse bone marrow-derived macrophage (BMM) primary cells at a range of SNIPR concentrations at 24 hours. (E) Fluorescence imaging of uptake of DiI-labelled SNIPR[pDNA-EGFP]nanoparticles in BMM cells 24 hours after treatment. (F) Fluorescence imaging of EGFP expression in BMM cells 6 days after treatment with DiI-labelled SNIPR[pDNA-EGFP]nanoparticles.

FIGS. 12-1, 12-2, 12-3, 12-4, 12-5, and 12-6: Comparison of cell viability versus transfection efficiency in cells treated with SNIPR or commercially available DNA transfection reagents.

FIGS. 13-1, 13-2, 13-3, 13-4, 13-5, and 13-6: Assessment of transfection efficiency mediated by SNIPR[pDNA-EGFP]nanoparticles after storage at different ambient temperatures (−20 C, 4 C, 25 C, or 37 C) for different amounts of time (0, 2, 4, 8, 16, or 32 weeks).

FIG. 14: Quantitation of immunogenicity of SNIPR nanoparticles in vitro.

FIG. 15: SNIPR nanoparticle maximum tolerable dose study in mice.

FIGS. 16A and 16B: Assessment of in vivo immunogenicity and efficacy of SNIPR-based Xavine™ nanoparticles in mice. (A) Quantitation of serum TNFa levels in treated mice six hours after the first dose. (B) Quantitation of splenic antigen-specific Foxp3+ regulatory T cells in treated mice.

FIGS. 17A-1, 17A-2, 17A-3, 17B-1, 17B-2, and 17B-3: Quantitation of tolerogenic phenotype induced by SNIPR-based Xavine™ nanoparticles in human dendritic cells in vitro.

FIGS. 18-1, 18-2, and 18-3: Biodistribution of SNIPR-based Xavine™ nanoparticles in mice.

FIGS. 19A and 19B: Quantitation of expression of MOG autoantigen in spleens of mice treated with SNIPR-based Xavine™ nanoparticles.

FIG. 20: Quantitation of secretion of IL-10 by splenocytes isolated from mice treated with SNIPR-based Xavine™ nanoparticles.

FIGS. 21A and 21B: Assessment of effect of SNIPR-based Xavine™ nanoparticle treatment on regulatory T cell populations in mice. (A) Quantitation of CD4+Foxp3+ Treg population in splenocytes of mice treated with two different SNIPR-based Xavine™ candidates. (B) Quantitation of MOG antigen specific Treg populations in mice treated with two different SNIPR-based Xavine™ candidates.

FIG. 22: Effect of MOG peptide restimulation on IFNg release by splenocytes isolated from mice treated with two different SNIPR-based Xavine™ candidates.

FIGS. 23A, 23B, 23C, 23D-1, and 23D-2: Assessment of mRNA delivery by SNIPR-023 variant nanoparticle. (A) Schematic of experimental design. (B) Flow cytometric quantitation of uptake of DiI-labelled SNIPR-023[mDNA-EGFP]nanoparticles in HEK-293 cells 24 hours after treatment. (C) Flow cytometric quantitation of transfection efficiency (percentage EGFP+ cells) in HEK-293 cells 24 and 72 hours after treatment with DiI-labelled SNIPR-023[mDNA-EGFP]. (D) Fluorescent imaging of uptake of DiI-labelled SNIPR-023[mDNA-EGFP]nanoparticles in HEK-293 cells 24 hours after treatment, and fluorescent imaging of resulting EGFP expression.

FIGS. 24A, 24B-1, 24B-2, and 24C: Various SNIPR lipid nanoparticles co-encapsulating plasmid DNA encoding mCherry and RNA encoding EGFP are efficiently taken up by and mediate dual transfection of HEK293 cells.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: in the compounds herein a C—H bond can be substituted with alkyl, lower alkyl, C₁₋₆ alkyl, C₆₋₁₀ aryl, C₆₋₁₀ heteroaryl, —NO₂, —NH₂, —N(R′R″)₂, —N(R′R″R′″)₃⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO3⁻M⁺, —PO3⁻M⁺, —COO⁻M⁺, —CF₂H, —CF₂R′, —CFH₃, and —CFR′R″ where R′, R″ and R′″ are C₁₋₁₀ alkyl or C₆₋₁₈ aryl groups; single letters (e.g., “n” or “o”) are 1, 2, 3, 4, or 5; percent, “parts of,” and ratio values are by weight; the term “polymer” includes “oligomer,” “copolymer,” “terpolymer,” and the like; molecular weights provided for any polymers refers to weight average molecular weight unless otherwise indicated; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim-as-a-whole.

The phrase “consisting essentially of” limits the scope of a claim to the specified materials or steps, plus those that do not materially affect the basic and novel characteristic(s) of the claimed subject matter.

The phrase “composed of” means “including,” “comprising,” or “consisting of.” Typically, this phrase is used to denote that an object is formed from a material.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100.

Throughout this application, where publications are referenced, the disclosures of these publications in their entireties are hereby incorporated by reference into this application to more fully describe the state of the art to which this invention pertains.

“Inverse micelle” means a micelle in which the nonpolar and polar phases have reversed roles and the orientation of surfactant molecules are inverted so that the head groups point into the enclosed volume containing the polar phase.

“Immune tolerance induction payload” means a payload (proteins, peptides, cytokines, polynucleotides, small molecules, and combinations thereof) that when taken up by one or more cell types of the immune system induces them to adopt a tolerogenic phenotype.

For a non-spherical object, “average diameter” means twice the average shortest distance from the center of mass of the object to the surface of the object. For spherical objects, diameter has the normal meaning. Sometimes “average diameter” is simply referred to a “diameter.”

### Abbreviations

“CL” means cardiolipin.
“DLS” means dynamic light scattering.
“EV” means extracellular vesicle.
“MVL” means multivesicular lipid nanoparticle.
“PC” means phosphatidylcholine.
“pDNA” means plasmid DNA.
“PE” means phosphatidylethanolamine.
“PEG” means polyethylene glycol.
“PS” means phosphatidylserine.
“PA” means phosphatidic acid.
“PI” means phosphatidylinositol.
“PG” means phosphatidylglycerol.
“siRNA” means silencing RNAs.
“SNIPR” means Sub-Nanoparticle Intracellular Payload Release.

In general, a multivesicular lipid nanoparticle composition that includes a plurality of multivesicular lipid nanoparticles is provided. Each multivesicular lipid nanoparticle includes a carrier lipid nanoparticle. The carrier lipid nanoparticle includes a carrier micelle (i.e., a carrier lipid nanoparticle micelle). The carrier micelle is composed of carrier lipids selected from the group consisting of phospholipids, lipids not including phosphorus, and combinations thereof, the carrier lipid nanoparticle having an average diameter of less than 1 micron. The multivesicular lipid nanoparticle composition also includes at least one sub-chamber lipid nanoparticle (e.g., micelle or inverse micelle) including a sub-chamber inverse micelle. The sub-chamber inverse micelle is composed of sub-chamber lipids selected from the group consisting of phospholipids, lipids not including phosphorus, and combinations thereof. Characteristically, the at least one sub-chamber lipid nanoparticle has an average diameter less than 100 nm with the carrier lipid nanoparticle encapsulating at least one sub-chamber lipid nanoparticle.

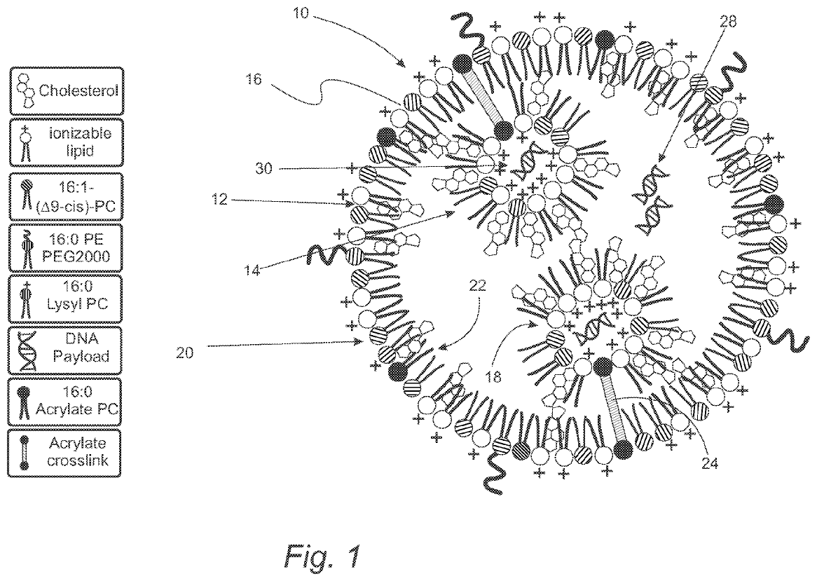

With reference to FIG. 1, a schematic illustration of the multivesicular lipid nanoparticle of the invention is provided. The multivesicular lipid nanoparticle designed herein is referred to as “SNIPR.” Multivesicular lipid nanoparticle 10 includes carrier micelle nanoparticle 12 and sub-chamber inverse micelles nanoparticles 14. Carrier micelle nanoparticle 12 is a sac-like structure that encapsulates at least one sub-chamber inverse micelle nanoparticle 14. In a refinement, carrier micelle nanoparticle 12 encapsulates a plurality of sub-chamber inverse micelle nanoparticles 14 (e.g. 1 to 100 or 2 to 10). In a variation, carrier micelle 12 is composed of carrier lipids selected from the group consisting of phospholipids, lipids not including phosphorus, and combinations thereof. In a refinement, carrier micelle nanoparticle 12 includes carrier phospholipid monolayer 16 that includes carrier phospholipids. Typically, carrier micelle nanoparticle 12 has a diameter (e.g., average diameter for a plurality of particles or individual particle diameter) less than 1 micron. In some refinements, carrier micelle nanoparticle 12 has a diameter (e.g., average diameter for a plurality of particles or individual particle diameter) less than, in increasing order of preference, 1 microns, 800 nm, 600 nm, 500 nm, 400 nm, or 300 nm. In a further refinement, carrier micelle nanoparticle 12 has a diameter (e.g., average diameter for a plurality of particles or individual particle diameter) greater than in increasing order of preference, 500 nm, 400 nm, 300 nm, 100 nm, 150 nm, or 200 nm. In a variation, sub-chamber inverse micelle 14 is composed of sub-chamber lipids selected from the group consisting of phospholipids, lipids not including phosphorus, and combinations thereof. In a refinement, sub-chamber inverse micelle nanoparticle 14 includes sub-chamber phospholipid monolayer 18 that includes sub-chamber phospholipids. Typically, sub-chamber inverse micelle nanoparticle 14 has a diameter (e.g., average diameter for a plurality of nanoparticles or individual nanoparticle diameter) equal to or less than about 100 nm. In a refinement, sub-chamber inverse micelle nanoparticle 14 has a diameter (e.g., average diameter for a plurality of nanoparticles or individual nanoparticle diameter) from about 20 nm to about 100 nm. In a refinement, sub-chamber inverse micelle nanoparticle has a diameter (e.g., average diameter for a plurality of nanoparticles or individual nanoparticle diameter) less than, in increasing order of preference, 150 nm, 125 nm, 100 nm, 90 nm, 80 nm, 70 nm, 60 nm, or 50 nm. In a further refinement, sub-chamber inverse micelle nanoparticle 14 has a diameter (e.g., average diameter for a plurality of particles or individual particle diameter) greater than, in increasing order of preference, 10 nm, 15, nm, 20 nm, 30 nm, 40 nm or 50 nm. Multivesicular lipid nanoparticle 10 includes an outer hydrophilic surface 20 and an inner hydrophobic surface 22. In a refinement, polyethylene glycol groups (PEG) are attached to the outer surface 20 of multivesicular micelle 10. In some refinements, PEG groups are attached to an inner surface of sub-chamber inverse micelles 14. In some refinements, cholesterol is incorporated into the inner hydrophobic surface 22 and sub-chamber inverse micelle nanoparticle 14. Characteristically, irreversible linkages 24 (e.g., via covalent acrylate polymerization) attach sub-chamber inverse micelle nanoparticle 14 to inner surface 22 of carrier micelle nanoparticle 12. In some refinements, reversible linkages 24 (e.g., disulfide linkage, pH sensitive linkages, electrostatic interactions, etc.) attach sub-chamber inverse micelle nanoparticle 14 to inner surface 22 of carrier micelle nanoparticle 12. Carrier micelle nanoparticle 12 can also encapsulate payloads 28. Similarly, sub-chamber inverse micelle nanoparticles 14 which are also sac-like can independently encapsulate payloads 30 which can be the same or different from payloads 28. It should be appreciated that the irreversible or reversible attachment of sub-chamber inverse micelles to the inner surface of the carrier micelle confers significant improvement in stability of the SNIPR multivesicular lipid nanoparticle compared to other lipid nanoparticle formulations, and is a critical feature of this innovation.

As set forth above, a therapeutic/diagnostic payload is encapsulated by at least one sub-chamber inverse micelle and/or the carrier micelle. In a variation, the therapeutic/diagnostic payload is selected from the group consisting of small molecule drugs, polynucleotides, proteins, peptides, ribonucleoproteins, and combinations thereof. Examples of therapeutic/diagnostic payload include fluorescent proteins, peptides, cytokines, antibodies, and combinations thereof. Examples of therapeutic immune tolerance induction payloads include immune tolerance induction proteins, peptides, cytokines, polynucleotides, small molecules, and combinations thereof. Examples of therapeutic payloads include autoantigen proteins, autoantigen peptides, polynucleotides encoding autoantigen proteins, polynucleotides encoding autoantigen peptides, and combinations thereof. Additional examples of therapeutic payloads include one or more fusion proteins containing one or more autoantigen epitopes, polynucleotides encoding fusion proteins containing one or more autoantigen epitopes, and combinations thereof. Additional examples of therapeutic payloads include immune tolerance induction proteins, peptides, cytokines, polynucleotides, small molecules, autoantigen proteins, autoantigen peptides, fusion proteins containing one or more autoantigen epitopes, polynucleotides encoding autoantigen proteins, polynucleotides encoding autoantigen peptides, polynucleotides encoding fusion proteins containing one or more autoantigen epitopes, and combinations thereof.

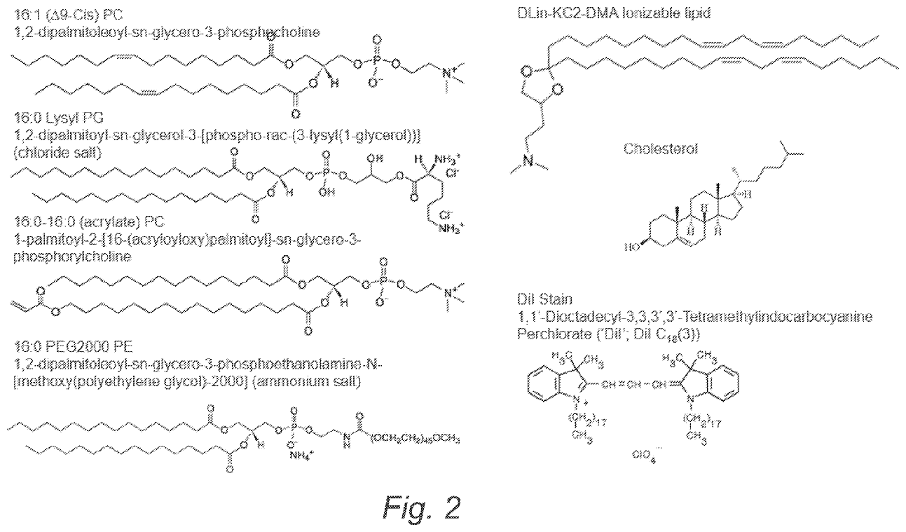

FIG. 2 illustrates the chemical structures of the components of one variation of the SNIPR multivesicular lipid nanoparticle.

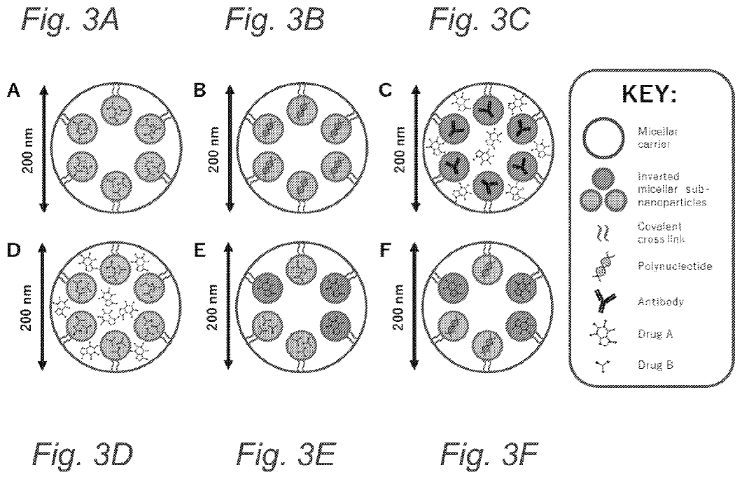

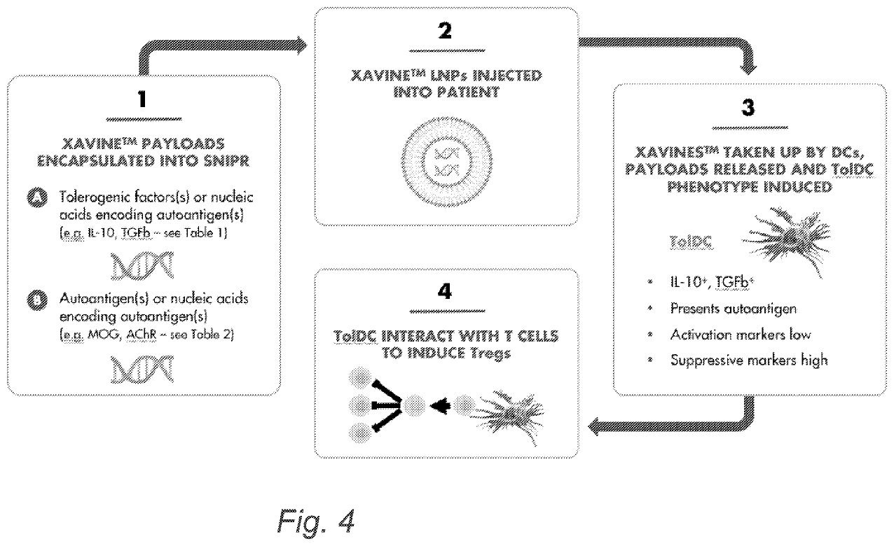

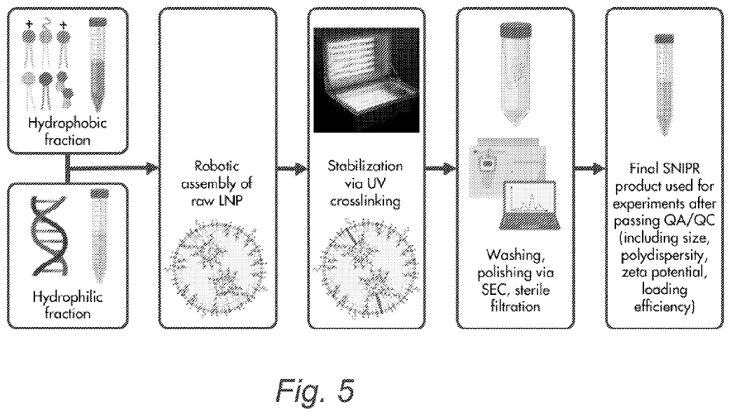

FIG. 3 illustrates a method and apparatus for delivering biomolecules (e.g. polynucleotides, small molecule drugs, or other biologics) of interest to healthy and/or disease state cells and/or tissues using the multivesicular lipid nanoparticle of FIG. 1. Advantageously, the multivesicular lipid nanoparticle provides a highly ordered, densely packed structure. In a refinement, the multivesicular lipid nanoparticle includes a novel combination of four component phospholipids, an ionizable lipid, cholesterol, and optionally a lipophilic dye (FIG. 2). Advantageously, the SNIPR multivesicular lipid nanoparticle can be used for delivery of the following therapeutic/diagnostic assets (i.e., payloads): (i) small molecule drugs, (ii) polynucleotides, such as plasmid DNA (pDNA), silencing RNAs (siRNA), messenger RNA (mRNA), (iii) and macromolecular proteins, such as fluorescent proteins, antigen proteins and allergens, cytokines, and antibodies; and combinations thereof. The shorthand notation, SNIPR[payload] means the multivesicular lipid nanoparticle encapsulating a payload “payload” which can be any compound, drug, polynucleotide, or other biologic that can be delivered by the multivesicular lipid nanoparticle.

FIG. 4 illustrates a method and apparatus for delivering biomolecules (e.g. polynucleotides, small molecule drugs, or other biologics) of interest to immune cells and/or tissues using the multivesicular lipid nanoparticle of FIG. 1 for the purpose of inducing immunological tolerance to one or more antigens in a subject identified as needing induction of immunological tolerance. The multivesicular lipid nanoparticles designed to encapsulate payloads intended to elicit therapeutic antigen-specific tolerance and deliver them to immune cells and/or tissues in this manner herein are referred to as “Xavines™”, “Xavine™ multivesicular lipid nanoparticles” or “Xavine™ MVLs”. Advantageously, the size (e.g., 10-200 nm) of the Xavine™ MVL is appropriate for uptake by dendritic cells by caveolin-mediated endocytosis. This gives the Xavine™ MVL the opportunity to fuse with the endosome and deliver its payload(s) to the cytoplasm of the dendritic cell (DC), wherein the payload(s) can elicit their therapeutic effect. In contrast, liposomal and lipid nanoparticle structures of other sizes are taken up by different cellular mechanisms (such as clathrin-mediated endocytosis or micropinocytosis) which results in their trafficking to and destruction by the lysosome of the cell, preventing the desired therapeutic outcome. Advantageously, the Xavine™ MVL can be used for delivery of the following therapeutic assets (i.e., payloads): (i) small molecule drugs, (ii) polynucleotides, such as plasmid DNA (pDNA), silencing RNAs (siRNA), messenger RNA (mRNA), (iii) and macromolecular proteins, such as antigen proteins and allergens, cytokines, and antibodies; and combinations thereof. This is advantageous because different combinations of payloads will be necessary to elicit the desired therapeutic effects to treat or prevent autoimmune diseases, allergies, and food sensitivities. This is central to the innovation, because it permits the Xavine™ MVLs to deliver one or more tolerizing signals to immune cells and/or tissues concomitantly with one or more payloads that induce antigen presenting cells to present the epitopes of one or more autoantigens. This contrasts with current technologies, which merely rely on antigen presenting cells defaulting to a tolerogenic state when they are forced to present autoantigen epitopes in the absence of proinflammatory signals, which may not be the case in patients. For example, the tolerogenic vaccine may be administered when the patient has an undetected viral or bacterial infection, which could then later cause autoantigen presentation in a proinflammatory environment, potentially worsening autoimmunity in that patient instead of improving it. Another example wherein current passive tolerogenic vaccines are likely to be administered in a strongly proinflammatory environment would be when they are administered to patients with active autoimmune disease—the very patient population they are intended to treat. Table 1 lists Xavine™ MVL payloads intended to actively induce a tolerogenic state in antigen presenting cells. Table 2 lists Xavine™ MVL payloads intended to induce the presentation of epitopes from autoantigens known to be pathogenic in the autoimmune diseases listed.

TABLE 1
Tolerogenic factor payloads that may be used in the
Xavine ™ multivesicular lipid nanoparticle.

| | Xavine ™ MVL |
|---|---|
| Tolerogenic Factor | Payload Type |
| Beta-glucan | |
| Interleukin 10 (IL-10) | mRNA, pDNA, protein |
| Interleukin 27 (IL-27) | mRNA, pDNA, protein |
| Tumor Growth Factor beta 1 | mRNA, pDNA, protein |
| (TGFb1) | |
| Hepatocyte growth factor | mRNA, pDNA, protein |
| (VGF) | |
| Vasoactive intestinal peptide | Peptide |
| (VIP) | |
| Vitamin D3 | Small molecule |
| Corticosteroids including | Small molecule |
| cortisone, prednisone, prednisolone, | |
| methylprednisolone, dexamethasone, | |
| betamethasone, hydrocortisone | |
| Rapamycin | Small molecule |
| Cyclosporine | Small molecule |
| Tacrolimus | Small molecule |
| Aspirin | Small molecule |
| Ligands of aryl hydrocarbon receptor | Small molecule |
| (AhR), including halogenated aromatic | |
| hydrocarbons (such as polychlorinated | |
| dibenzodioxins, dibenzofurans and | |
| biphenyls); polycyclic aromatic | |
| hydrocarbons (such as 3- | |
| methylcholanthrene, benzo[a]pyrene, | |
| benzanthracenes and benzoflavones); | |
| indigo dye; indirubin; bilirubin; lipoxin | |
| A4; prostaglandin G; carbidopa; | |
| indolocarbazole | |

TABLE 2
Autoantigen expression and presentation payloads that may
be used in the Xavine ™ multivesicular lipid nanoparticle.

| | | Xavine ™ MVL |
|---|---|---|
| | | Payload Types to |
| | | Induce Epitope |
| | | Presentation of this |
| Disease | Autoantigen | Autoantigen |
| Multiple sclerosis | Myelin basic protein (MBP); myelin | mRNA, pDNA, peptide, |
| | oligodendrocyte glycoprotein (MOG); | full length protein |
| | myelin proteolipid protein (PLP); | |
| | myelin-associated glycoprotein (MAG); | |
| | myelin-associated oligodendrocyte basic | |
| | protein (MOBP); 2′,3′-Cyclic-nucleotide | |
| | 3′-phosphodiesterase (CNPase); S100 | |
| | calcium-binding protein B (S100β); | |
| | transaldolase (TALDO1) | |
| MOG antibody disease | myelin oligodendrocyte glycoprotein | |
| | (MOG) | |
| Myasthenia gravis | Nicotinic acetylcholine receptor | mRNA, pDNA, peptide, |
| | (nAChR); muscle-specific tyrosine | full length protein |
| | kinase (MuSK); lipoprotein-related | |
| | protein 4 (LRP4) | |
| Diabetes mellitus type 1 | Insulin; islet antigen 2 (IA-2); glutamic | mRNA, pDNA, peptide, |
| | acid decarboxylase 65-kilodalton | full length protein |
| | isoform (GAD65); glutamic acid | |
| | decarboxylase 67-kilodalton isoform | |
| | (GAD67); solute carrier family 30 zinc | |
| | transporter 8 (ZnT8); islet-specific | |
| | glucose-6-phosphatase catalytic subunit- | |
| | related protein (IGRP); Chromogranin A | |
| Neuromyelitis optica | Aquaporin-4 (AQP-4); myelin | mRNA, pDNA, peptide, |
| | oligodendrocyte glycoprotein (MOG) | full length protein |
| Autoimmune | N-methyl-D-aspartate receptor | mRNA, pDNA, peptide, |
| encephalitides to | (NMDAR); α-amino-3-hydroxy-5- | full length protein |
| membrane antigens | methyl-4-isoxazolepropionic acid | |
| | receptor (AMPAR); gamma | |
| | aminobutyric acid isotype A receptor | |
| | (GABAAR); gamma aminobutyric acid | |
| | isotype B receptor (GABABR); glycine | |
| | receptor (GlyR); dipeptidyl peptidase | |
| | like protein-6 (DPPX); glutamate | |
| | receptor, ionotropic, kainate 1 (GRIK1 | |
| | also known as GluR5); voltage-gated | |
| | potassium channel complex (VGKC- | |
| | complex) | |
| Autoimmune | paraneoplastic antigen Ma1 (PNMA1); | mRNA, pDNA, peptide, |
| encephalitides to | paraneoplastic antigen Ma2 (PNMA2); | full length protein |
| intracellular antigens | zic family member 4 (Zic4); glutamic | |
| | acid decarboxylase 65-kilodalton | |
| | isoform (GAD65); glutamic acid | |
| | decarboxylase 67-kilodalton isoform | |
| | (GAD67); collapsin response-mediator | |
| | protein-5 (CRMP5); Amphiphysin | |
| Rheumatoid arthritis | Modified citrullinated vimentin (MCV); | mRNA, pDNA, peptide, |
| | collagen type II (COL2A1); 65-kDa | full length protein |
| | heat-shock protein (HSP65); cartilage | |
| | glycoprotein-39 (HC-gp39); aggrecan | |
| | G1 (ACAN); immunoglobulin heavy | |
| | constant gamma 1 (IGHG1); acetyl-CoA | |
| | acetyltransferase 2 (ACAT2); aldolase, | |
| | fructose-bisphosphate A (ALDOA); | |
| | carcinoembryonic antigen cell adhesion | |
| | molecule 1 (CEACAM1); eukaryotic | |
| | translation elongation factor 1 gamma | |
| | (EEF1G); inosine monophosphate | |
| | dehydrogenase 1 (IMPDH1); keratin 19 | |
| | (KRT19); lactate dehydrogenase-B | |
| | (LDHB); macrophage migration | |
| | inhibitory factor (MIF); transketolase | |
| | (TKT); slow muscle alpha (α)- | |
| | tropomyosin (TPM3); vimentin (VIM); | |
| | calcium/calmodulin dependent protein | |
| | kinase II gamma (CAMK2G); calcium | |
| | regulated heat stable protein 1 | |
| | (CARHSP1); eukaryotic translation | |
| | elongation factor 1 delta (EEF1D); | |
| | interferon induced protein 35 (IFI35); | |
| | interleukin enhancer binding factor 2 | |
| | (ILF2); protein kinase CAMP- | |
| | dependent type I regulatory subunit | |
| | alpha (PRKARIA); proteasome | |
| | activator subunit 3 (PSME3); ribosomal | |
| | protein lateral stalk subunit P1 (RPLP1); | |
| | RUN and FYVE domain containing 1 | |
| | (RUFY1); small RNA binding | |
| | exonuclease protection factor La (SSB); | |
| | signal transducer and activator of | |
| | transcription 1 (STAT1); tropomyosin 1 | |
| | (TPM1) | |
| | Citrullinated antigens; carbamylated | Peptide, full length |
| | antigens | protein |
| Hashimoto's | thyroglobulin (Tg); thyroid peroxidase | mRNA, pDNA, peptide, |
| autoimmune thyroiditis | (TPO); thyrotropin receptor (TSHR) A- | full length protein |
| | subunit | |
| Celiac disease | Tissue transglutaminase (TG2) | mRNA, pDNA, peptide, |
| | | full length protein |
| Graves' disease | Thyrotropin receptor (TSHR) | mRNA, pDNA, peptide, |
| | | full length protein |
| Vitiligo | gamma-enolase (ENO2); o-enolase | mRNA, pDNA, peptide, |
| | (ENO1); heat-shock protein 90 | full length protein |
| | (HSP90); osteopontin (SPP1); ubiquitin- | |
| | conjugating enzyme E2 V1 (UBE2V1); | |
| | eukaryotic translation-initiation factor 2 | |
| | (eIF2); ras-related protein rab-38 | |
| | (RAB38) | |
| Rheumatic fever | cardiac beta-myosin heavy chain | mRNA, pDNA, peptide, |
| | (MYH7); vimentin (VIM) | full length protein |
| Pernicious | gastric H+/K+ ATPase (ATP4A) | mRNA, pDNA, peptide, |
| anemia/atrophic gastritis | | full length protein |
| Alopecia areata | trichohyalin (TCHH); tyrosinase-related | mRNA, pDNA, peptide, |
| | protein-2 (TRP-2) | full length protein |
| Immune | Platelet glycoprotein (GP) IIb/IIIa | mRNA, pDNA, peptide, |
| thrombocytopeni | (ITGA2B); Glycoprotein Ib complex | full length protein |
| purpura | components (GP1b-alpha, GP1b-beta), | |
| | GP1X, GPV) | |
| Psoriasis | Cathelicidin antimicrobial peptide | mRNA, pDNA, peptide, |
| | (CAMP); a disintegrin-like and | full length protein |
| | metalloprotease domain containing | |
| | thrombospondin type 1 motif-like 5 | |
| | (ADAMTSL5); phospholipase A2 group | |
| | IVD (PLA2G4D); keratin 17 (KRT17) | |

As set forth above, the carrier micelle and sub-chamber inverse micelle can be independently composed of sub-chamber lipids selected from the group consisting of phospholipids, lipids not including phosphorus, and combinations thereof. In a variation, the carrier micelle and/or the at least one sub-chamber lipid nanoparticle include a lipid described by the following formula:

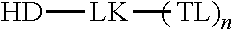

wherein:
HD is a head group;
LK is a linking group;
TL is a tail group; and
n is an integer representing the number of tail groups. In a refinement, n can be 1, 2, 3, or 4. It should be appreciated that when there are more than 1 tail group, the tail groups can be the same or different. Typically, the tail groups include branched or unbranched C₆₋₂₅ aliphatic chains. In some refinements, the tail groups include one or more carbon-carbon double bonds with can be cis or trans. In some refinement, the lipids include multiple aliphatic chains that are symmetric to each other. In some refinements, one or more carbon atoms in the aliphatic chains can be replaced with O, N, S, or a carbonyl. In some refinements, the aliphatic chains can include one or more ester groups. In a refinement, wherein when there are more than one tail groups, the tail groups can be the same or different.

In a refinement, the head group HD includes a moiety selected from the group consisting of:

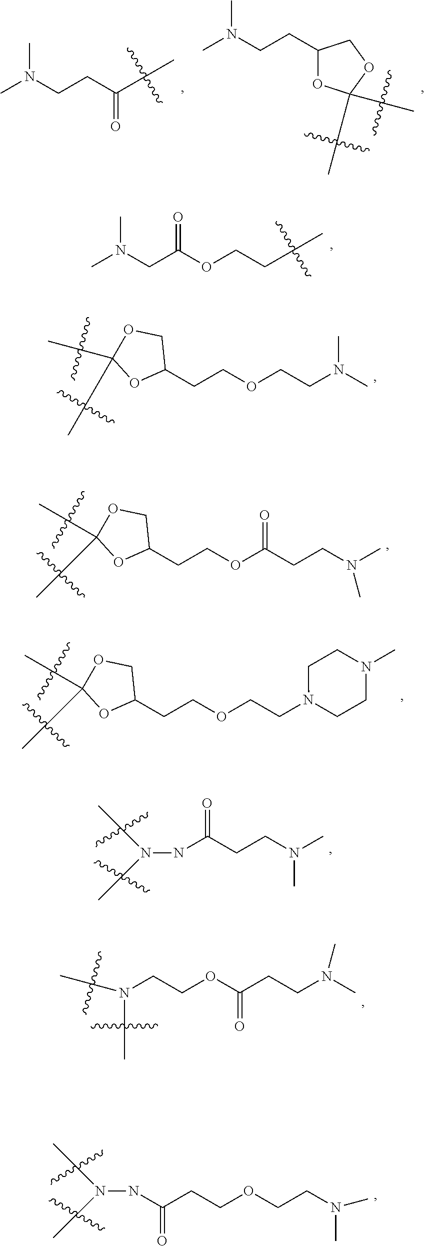

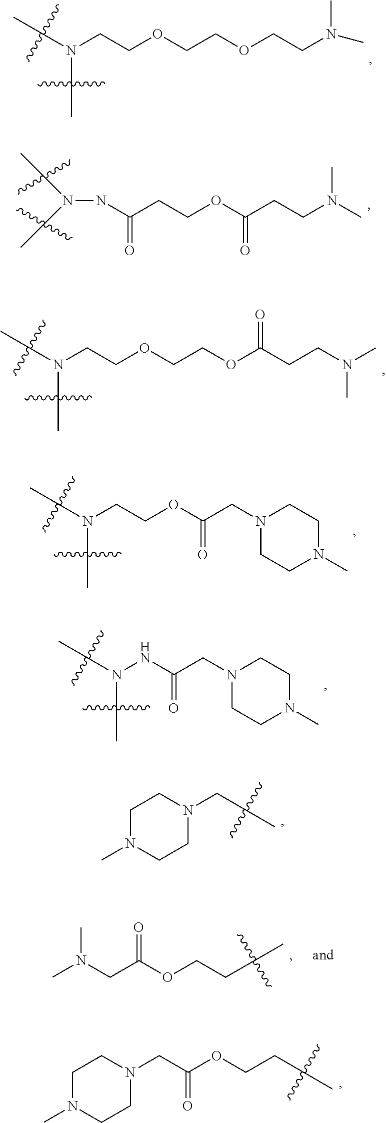

where the wiggly line represents the point of attachment to the portion of the lipid structure.

Examples of lipids include:

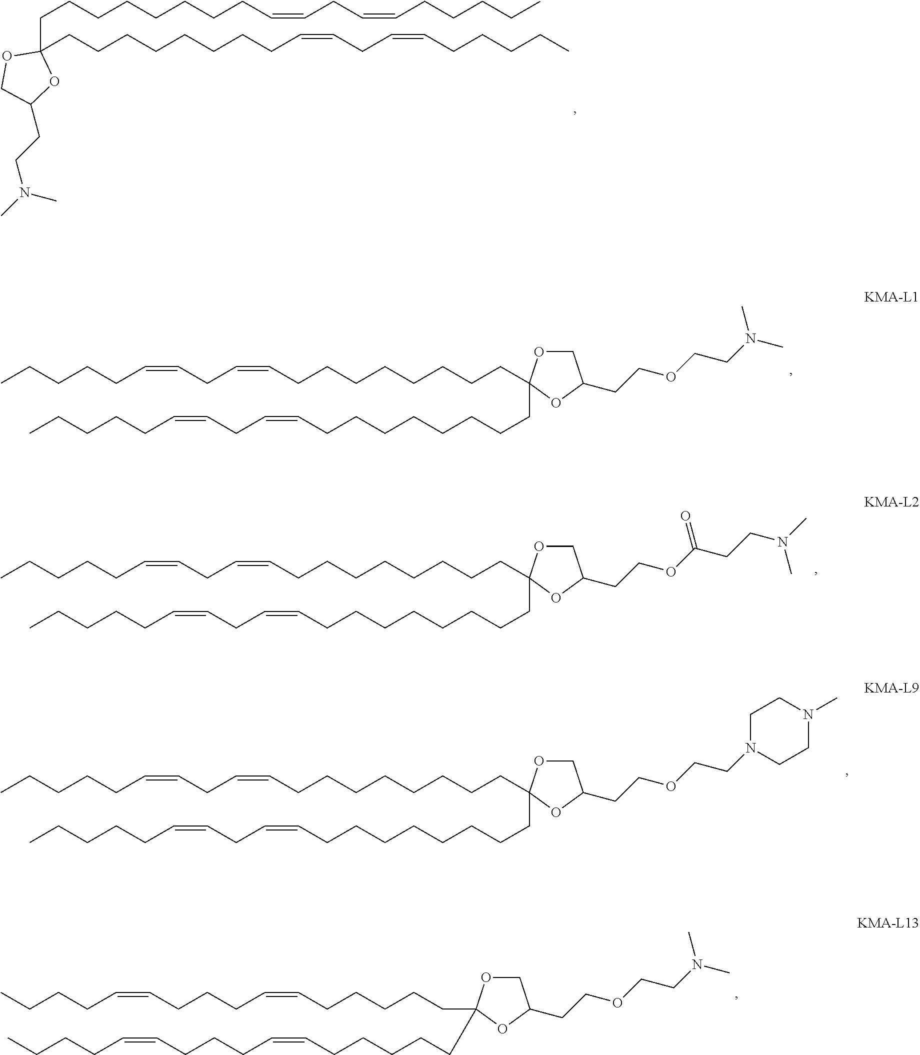

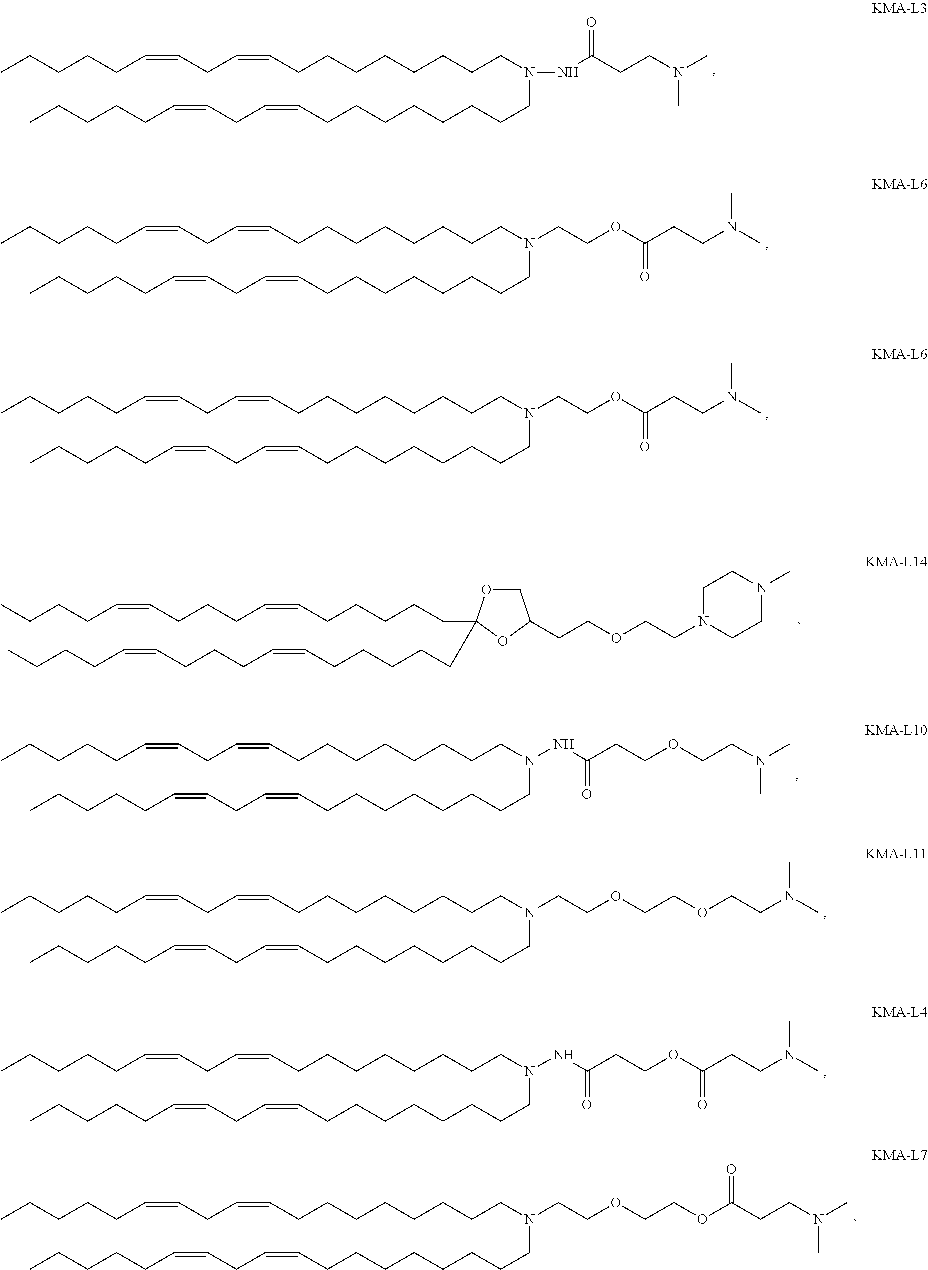

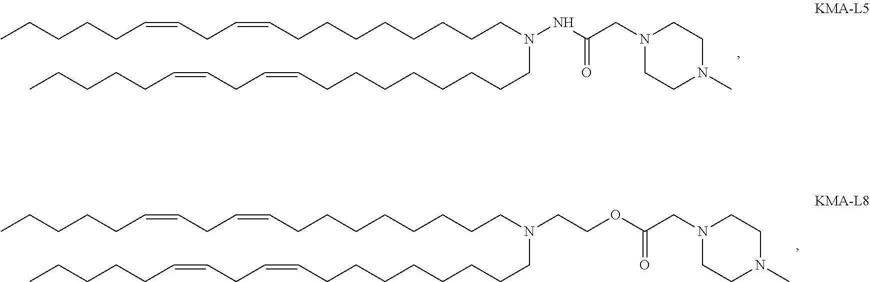

and combinations thereof.

As set forth above, the size of these lipid nanoparticles is controlled by the tail group length of the phospholipids used to make up the lipid monolayers. The following formula provides the generic structure of a phospholipid:

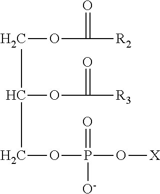

where O═C—R₂ and O═C—R₃ are tail groups; and X is the head group substituent (e.g., PC, PE, PS, PA, PI, PG or CL). As set forth above, the tail groups can include branched or unbranched C₆₋₂₅ aliphatic chains. Therefore, R₂ and R₃ are independently C₆₋₂₅ aliphatic chains. In some refinements, the tail groups include one or more carbon-carbon double bonds with can be cis or trans. In some refinement, the lipids include multiple aliphatic chains that are symmetric to each other. In some refinements, one or more carbon atoms in the aliphatic chains can be replaced with O, N, S, or a carbonyl. In some refinements, the aliphatic chains can include one or more ester groups. In some refinements, a lipid nanoparticle phospholipid monolayer is a combination of phospholipids having from 14 to 17 carbons. At least some of the tail groups for lipid nanoparticle phospholipids can include a double bond, and in particular, a cis double bond. Moreover, at least some of the tail groups for lipid nanoparticle phospholipids can include photoactive groups that can be crosslinked when the phospholipids are included in the phospholipid monolayers. Examples of such photoactive groups include moieties with ethylenic unsaturation such as acrylate groups. Moreover, at least some of the headgroups for lipid nanoparticle phospholipids can include ionizable structures that adopt a positive charge in low pH environments (such as acidic buffers or within the endosome of the cell), but have a neutral charge in neutral pH environments (such as neutral buffers or within the extracellular environment within the body) when the phospholipids are included in the phospholipid bilayers. Moreover, at least some of the headgroups for lipid nanoparticle phospholipids can include structures that are positively charged regardless of the pH of the environment when the phospholipids are included in the phospholipid bilayers.

Chemical modifications to the tail groups of phospholipids can drive and/or stabilize inter-bilayer association between compartments in the MVLs. This key architectural mechanism stabilizes SNIPR multivesicular lipid nanoparticles with encapsulated payloads, prolonging the time until release. A key novelty of our innovation is in the organization of the principle apparatus, wherein the outer surface of the sub-chamber inverse micelle nanoparticles are covalently linked to the inner surface of the carrier micelle in this manner. In the principal apparatus, in particular, the bilayer composition is stabilized by a network of UV conjugated tail group covalent linkages. As a result, the lipid nanoparticle lipid bilayer surface is composed of rafts of bilayers shifting based on relieving the structural strain of the particle. In nature, cells undergo this process via the actions of transmembrane protein complexes to enhance structural stability, whereas our system achieves a similar effect based on a series of covalent linkages between the tail groups of phospholipids.

The lipid nanoparticles include phospholipids or residues thereof. The lipids used in forming the lipid nanoparticle bilayers are listed in Table 3. In this context the term “residues” referred to reaction products (e.g., crosslinking) of these phospholipids when such groups are included in a phospholipid bilayer.

TABLE 3
Phospholipids used in the multivesicular lipid nanoparticle.

| | | Carbon | | | | |
|---|---|---|---|---|---|---|
| Common | | Length (tail | | Functional | Amount | Range |
| Name | IUPAC Name | group) | Saturation | Group | (%) | (%) |
| 16:1 (Δ9-Cis) | 1,2-dipalmitoleoyl- | 16 | Un- | N/A | 10.0 | 9.0-95.0 |
| PC | sn-glycero-3- | | saturated, | | | |
| | phosphocholine | | cis | | | |
| 16:0 Lysyl | 1,2-dipalmitoyl-sn- | 16 | Saturated | N/A | 1.0 | 0.0-1.0 |
| PG | glycero-3-[phospho- | | | | | |
| | rac-(3-lysyl(1- | | | | | |
| | glycerol))] | | | | | |
| | (chloride salt) | | | | | |
| 16:0-16:0 | 1-palmitoyl-2-[16- | 16 | Saturated | Acrylate | 1.0 | 1.0-25.0 |
| (acrylate) PC | (acryloyloxy)palmitoyl]- | | | | | |
| | sn-glycero-3- | | | | | |
| | phosphorylcholine | | | | | |
| 16:0 | 1,2-dipalmitoyl-sn- | 16 | Saturated | N/A | 1.5 | 1.5 |
| PEG•2000 | glycero-3- | | | | | |
| PE | phosphoethanolamine-N- | | | | | |
| | [methoxy(polyethylene | | | | | |
| | glycol)-2000] | | | | | |
| | (ammonium salt) | | | | | |
| Dlin-KC2- | 2-[2,2-bis[(9Z,12Z)- | 19 | Un- | N/A | 50.0 | 4.0-50.0 |
| DMA; Dlin- | octadeca-9,12-dienyl]- | | saturated, | | | |
| MC3-DMA; | 1,3-dioxolan-4-yl]- | | cis | | | |
| examples of | N,N-dimethylethanamine; | | | | | |
| ionizable | (6Z,9Z,28Z,31Z)- | | | | | |
| lipids set | Heptatriaconta- | | | | | |
| forth above | 6,9,28,31-tetraen-19-yl | | | | | |
| or | 4-(dimethylamino)butanoate; | | | | | |
| combinations | Ionizable Lipids set forth | | | | | |
| thereof_{—} | above or combinations | | | | | |
| | thereof | | | | | |
| Cholesterol | Cholest-5-en-3β-ol | | | | 36.5 | 0-38.5 |
| DiI Stain | (2Z)-2-[(E)-3- | 18 | Saturated | N/A | 0.0 | 0.0-0.1 |
| | (3,3-dimethyl-1- | | | | | |
| | octadecylindol-1-ium-2- | | | | | |
| | yl)prop-2-enylidene]- | | | | | |
| | 3,3-dimethyl-1- | | | | | |
| | octadecylindole; | | | | | |
| | perchlorate | | | | | |

The principal apparatus of the lipid nanoparticle is composed of 4 unique phospholipids, an ionizable lipid, cholesterol, and an optional lipophilic dye, organized in a molar ratio to promote a monodispersed distribution (100-300 nm in diameter) of lipid nanoparticles. The organization of the principle apparatus is composed of the following components: (1) 1,2-dipalmitoleoyl-sn-glycero-3-phosphocholine, (2) 1,2-dipalmitoyl-sn-glycero-3-[phospho-rac-(3-lysyl(1-glycerol))](chloride salt), (3) 1-palmitoyl-2-[16-(acryloyloxy)palmitoyl]-sn-glycero-3-phosphorylcholine, (4) 1,2-dipalmitoyl-sn-glycero-3-phosphoethanolamine-N-[methoxy(polyethylene glycol)-2000](ammonium salt), (5) 2,2-dilinoleyl-4-dimethylaminoethyl-[1,3]-dioxolane, (6) Cholest-5-en-3β-ol, and (7) (2Z)-2-[(E)-3-(3,3-dimethyl-1-octadecylindol-1-ium-2-yl)prop-2-enylidene]-3,3-dimethyl-1-octadecylindole; perchlorate. In a variation, residues thereof are present in an amount from 9 mole percent to 95 mole percent of the total amount of the phospholipids; 1,2-dipalmitoyl-sn-glycero-3-[phospho-rac-(3-lysyl(1-glycerol))](chloride salt) or residues thereof are present in an amount from about 0 mole percent to about 1 mole percent of the total amount of the phospholipids; 1-palmitoyl-2-[16-(acryloyloxy)palmitoyl]-sn-glycero-3-phosphorylcholine or residues thereof are present in an amount from about 1 mole percent to about 10 mole percent of the total amount of the phospholipids; 1,2-dipalmitoyl-sn-glycero-3-phosphoethanolamine-N-[methoxy(polyethylene glycol)-2000](ammonium salt) or residues thereof are present in an amount from about 0 mole percent to about 1.5 mole percent of the total amount of the phospholipids; 2,2-dilinoleyl-4-dimethylaminoethyl-[1,3]-dioxolane or residues thereof are present in an amount from about 4 mole percent to about 50 mole percent of the total amount of the phospholipids; cholest-5-en-30-ol or residues thereof are present in an amount from about 0 mole percent to about 38.5 mole percent of the total amount of the phospholipids; (2Z)-2-[(E)-3-(3,3-dimethyl-1-octadecylindol-1-ium-2-yl)prop-2-enylidene]-3,3-dimethyl-1-octadecylindole; perchlorate or residues thereof are present in an amount from about 0 mole percent to about 0.1 mole percent of the total amount of the phospholipids; and (2E)-3-octadecyl-2-[(E)-3-(3-octadecyl-1,3-benzoxazol-3-ium-2-yl)prop-2-enylidene]-1,3-benzoxazole; perchlorate or residues thereof are present in an amount from about 0 mole percent to about 0.1 mole percent of the total amount of the phospholipids.

The organization and stability of the lipid nanoparticle apparatus is promoted by the crosslinking of phospholipid tail groups (e.g., 16:0 acrylate PC phospholipid tail groups) through reactive terminal olefin on at least a subset of the phospholipid tail groups, upon the formation of lipid nanoparticles in an aqueous solution. The crosslinking of tail groups is driven by a photo-initiator (e.g. 2-hydroxy-4′(2-hydroxyethoxy)-2-methlipropiophenone [HEM]), which forms a radical intermediate at a carbon atom in the reactive terminal olefin, in particular, the carbon atom furthest from the carbonyl group (e.g., carbon-16 for 16:0 acrylate PC, reactive terminal olefin) upon exposure to UV light. The radical intermediate reacts with other neighboring tail groups, containing a terminal olefin, in a chain reaction, until there are no more reactive terminal olefins. An essential aspect of this organization is the ratio of phospholipids with reactive terminal olefins to phospholipids without; too many will result in too much crosslinking and therefore a lipid nanoparticle that cannot release its payload, too few will result in an unstable lipid nanoparticle that releases its payload prematurely. Also relevant, the organization and stability of the lipid nanoparticle to encapsulate payloads is promoted by enhancements in the charge character of the 16:0 lysyl PG phospholipid head group and the ionizable lipid (for example, D-Lin-KC2-DMA). During the formation of the lipid nanoparticle, positively charged 16:0 lysyl PG head groups and D-Lin-KC2-DMA ionizable head groups are drawn to negatively charged payloads (such as the phosphate backbones of nucleic acids). As the lipid nanoparticle monolayers associate, payload molecules are trapped, or encapsulated, in the newly formed lipid nanoparticle. As a result, the lipid nanoparticle promotes high-encapsulation efficiencies (>60%) of payload molecules. Of central importance to this innovation is the ratio of permanently positively charged headgroups (on 16:0 lysyl PG phospholipids) to temporarily positively charged headgroups (on D-Lin-KC2-DMA lipids). Without sufficient positive charge, lipid nanoparticles cannot initiate and maintain association with negatively charged payloads such as nucleic acids during assembly, nor can they fuse efficiently with the endosome for appropriate payload release. However, lipid nanoparticles that have a high and permanent positive charge are toxic, and can associate with the negatively charged cellular membrane which facilitates uptake by mechanisms that favor their destruction prior to payload release. The presence of ionizable headgroups (for example, on D-Lin-KC2-DMA lipids) allows the temporary induction of a strong positive charge during manufacture (facilitating payload encapsulation) and also within the endosome (facilitating endosornal escape and payload delivery to the cytoplasm), while also allowing the lipid nanoparticle to adopt a neutral charge in other environments such as in the body (thereby minimizing toxicity and destructive uptake by cells). The presence of headgroups that are permanently positively charged (on 16:0 lysyl PG phospholipids) maintains the association between the lipid bilayers and the negatively charged payloads regardless of the local environment, and also increases the likelihood that the lipid bilayers will adopt a hexagonal formation within the endosome, promoting endosomal fusion and payload release into the cytoplasm. The presence of cholesterol in the lipid nanoparticle lipid bilayer also facilitates endosomal fusion and cytoplasmic payload delivery.

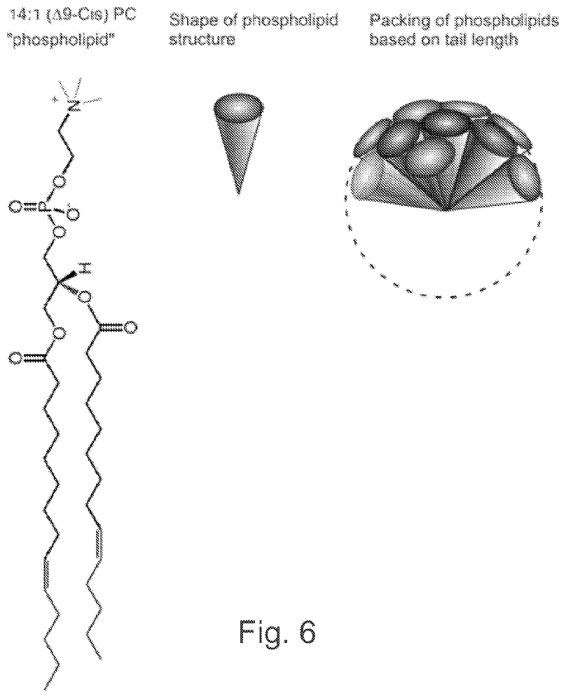

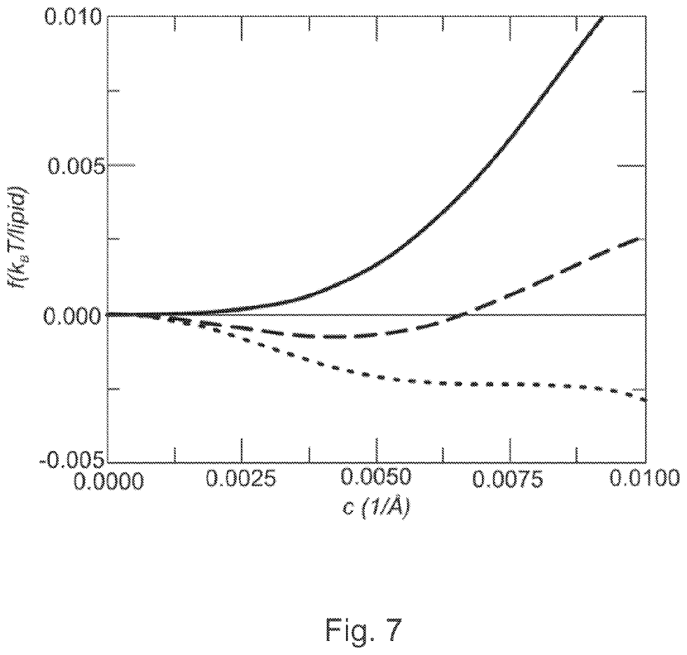

The structural organization of the lipid nanoparticle's phospholipids are relevant to certain embodiments of the invention. In particular, the length of the tail group drives the curvature, and therefore, diameter of the liposomal nanoparticle. Phospholipids-containing shorter carbon-length tail groups are susceptible to forming shorter angles with other similar phospholipids (FIG. 6). As the non-polar tail groups become shorter, the curvature of the bilayer is driven by the opposing forces of the head group.³ Another key aspect of the structural organization is the permeability of the lipid nanoparticle, which is driven by the cis-, or trans-, confirmation of the unsaturated phospholipids. From Giliespe et al. and first principles, these parameters control the release rate of payloads from the lipid nanoparticle.⁴ Another key aspect of the structural organization is the covalent addition of water-soluble poly (ethylene glyco) (PEG) to the surface of the lipid nanoparticles. As shown in FIG. 7, the curvature of a PEG-functionalized bilayer is energetically stable compared to bilayers, not functionalized with PEG.⁵

In another embodiment, a method for forming the multivesicular lipid nanoparticle composition set forth above is provided. The method includes a step of forming a plurality of multivesicular lipid nanoparticles where the plurality of multivesicular lipid nanoparticles have an average nanoparticle diameter less than about 1 micron. The plurality of multivesicular lipid nanoparticles are formed from the combination of two MVL-forming compositions that include one or more payloads. The first MVL-forming composition comprises the hydrophobic components of the MVL composition (e.g., the phospholipids and any hydrophobic payload(s) are combined in a nonaqueous solvent such as ethanol, methanol, DMSO, or DMF). In a refinement, an aqueous fraction of the MVL-forming composition includes one or more additional hydrophilic therapeutic/diagnostic payloads and/or an organic fraction of sub-chamber lipid-forming composition includes one or more hydrophobic therapeutic/diagnostic payload(s). The second MVL-forming composition comprises the hydrophilic components of the MVL composition (typically one or more hydrophilic payloads) in an aqueous solution of appropriate pH and molarity to elicit a positive charge on ionizable lipid headgroups and facilitate spontaneous formation of the multivesicular lipid nanoparticles when mixed with the first MVL composition. In one variation, the first multivesicular lipid nanoparticle-forming composition includes 1,2-dipalmitoleoyl-sn-glycero-3-phosphocholine, 1,2-dipalmitoyl-sn-glycero-3-[phospho-rac-(3-lysyl(1-glycerol))](chloride salt), 1-palmitoyl-2-[16-(acryloyloxy)palmitoyl]-sn-glycero-3-phosphorylcholine, 1,2-dipalmitoyl-sn-glycero-3-phosphoethanolamine-N-[methoxy(polyethylene glycol)-2000](ammonium salt), 2,2-dilinoleyl-4-dimethylaminoethyl-[1,3]-dioxolane, Cholest-5-en-3β-ol, and (2Z)-2-[(E)-3-(3,3-dimethyl-1-octadecylindol-1-ium-2-yl)prop-2-enylidene]-3,3-dimethyl-1-octadecylindole; perchlorate which are mixed together typically in a solvent. One or more hydrophobic payloads are optionally combined with the first multivesicular lipid nanoparticle-forming composition. In this variation, the second multivesicular lipid nanoparticle-forming composition includes a 25 mM acetate solution (prepared with acetic acid and sodium acetate to a pH of 4) and optionally one or more hydrophilic payload(s). The first and second multivesicular lipid nanoparticle-forming compositions are then mixed in a 1:3 ratio via a mixing robot such as a NanoAssemblr device (Precision Nanosystems, Inc.). The raw multivesicular lipid nanoparticle are then crosslinked with actinic radiation (e.g., UV light), washed, concentrated, and filter sterilized prior to use.

In a variation, Xavine™ nanoparticles are made using a modified version of the method set forth above. In this variation, the payload is one or more hydrophilic biomolecules that induce presentation of one or more antigens on the cell surface of treated antigen presenting cells. In a further variation, the first payload is one or more hydrophilic biomolecules that induce presentation of one or more antigens on the cell surface of treated antigen presenting cells, and the second payload is one or more hydrophilic biomolecules that induce a tolerogenic phenotype in antigen presenting cells (e.g., pDNA encoding IL-10, TGFb1, or IL-27), and/or one or more hydrophobic biomolecules that induce a tolerogenic phenotype in antigen presenting cells (e.g. rapamycin).

The following examples illustrate the various embodiments of the present invention. Those skilled in the art will recognize many variations that are within the spirit of the present invention and scope of the claims.

### Methods

### Solutions:

The following solutions may be made and stored ahead of time, as necessary:
1. 1× Phosphate Buffer Solution (1×PBS)
2. 125 mM Acetate Buffer, pH 4.0 (5×)
a. In 180 mL water, dissolve 465 mg Sodium Acetate
b. To Sodium Acetate solution, add 1109 μL of glacial Acetic Acid (17.4 M)
c. Adjust pH to 4.0 with glacial Acetic Acid or 10 N Sodium Hydroxide
d. Dilute solution to 200 μL.
3. 25 mM Acetate Buffer, pH
a. Dilute 10 mL of the 5× Acetate Buffer solution to 50 mL with water.

Lipids Stocks (prepared in Ethanol; to be stored at −20°)
4. 100 mg/mL 16:1-(Δ9-cis)-PC Lipid
5. 5 mg/mL 16:0 lysyl-PG Lipid
6. 10 mg/mL 16:0-16:0 Acrylate PC Lipid
7. 10 mg/mL 16:0 PEG2000 P E Lipid
8. 100 mg/mL D-Lin-KC2-DMA Ionizable Lipid
9. 10 mg/mL Cholesterol
10. 1 mg/mL DiI—1,1′-Dioctadecyl-3,3,3′,3′-Tetramethylindocarbocyanine Perchlorate (‘DiI’; DiI C₁₈(3))

The following solutions are to be made immediately prior to use:
11. 1% (v/v) Tetramethylethylenediamine (TEMED)
a. Add 3 μL of TEMED to 297 μL of 25 mM Sodium Acetate Buffer.
b. Vortex thoroughly and store at room temperature away from light.
12. 0.2% (w/v) 2-Hydroxy-4′-(2-hydroxyethoxy)-2-methlpropiophenone (HEM)
a. Weigh 10 mg of HEM and dissolve in 500 μL of Ethanol.
b. Dilute a 10 μL aliquot to 100 μL in Ethanol
c. Vortex thoroughly and store at room temperature away from light.

### Starting Material Preparation:

The lipid preparation is determined according to the following table:

| | | μMoles | | | Concentration | |
|---|---|---|---|---|---|---|
| | Molar Ratio | (Total = 8.3 μmol) | MW (g/mol) | Mass of lipid | (mg/mL, EtOH) | Volume (μL) |
| 16:1-(Δ9-cis)-PC | 10.0 | 0.83 | 730.007 | 605.91 | 100 | 6.06 |
| 16:0 Lysyl PG | 1.0 | 0.083 | 924.064 | 76.70 | 5 | 15.33 |
| 16:0-16:0 Acrylate PC | 1.0 | 0.083 | 804.086 | 66.74 | 10 | 6.67 |
| 16:0 PEG2000 PE | 1.5 | 0.125 | 2749.391 | 343.67 | 10 | 34.2 |
| D-Lin-KC2-DMA | 50.0 | 4.15 | 642.09 | 2664.67 | 100 | 26.6 |
| Cholesterol | 36.4 | 3.02 | 386.65 | 1167.68 | 10 | 116.8 |
| DiI | 0.1 | 0.0083 | 933.89 | 7.75 | 1 | 7.75 |

13. To a microcentrifuge tube, add the following volumes from the lipid stocks. (Note, though only 375 μL will be used in the final product, the operation of the Precision Nanosystems NanoAssembler requires an excess volume.)
a. 323 μL of Ethanol
b. 12.12 μL of 16:1-(Δ9-cis)-PC Lipid
b. 30.7 μL of 16:0 PG Lipid
c. 13.34 μL of 16:0-16:0 Acrylate PC Lipid
d. 68.5 μL of 16:0 PEG2000 P E Lipid
e. 53.5 μL of D-Lin-KC2-DMA Ionizable Lipid
f. 234.0 μL of Cholesterol.
g. 15.5 μL of DiI,
14. Vortex the lipid mixture thoroughly, and store at room temperature for three hours away from light.
15. Under aseptic conditions, prepare a 1500 μL plasmid “payload” solution. (Note, though only 1125 μL will be used in the final product, the operation of the Precision Nanosystems NanoAssembler requires an excess volume.)
a. The final concentration of the plasmid must be 203 g/mL in order to yield a 6:1 N/P ratio, and the final concentration of the Acetate buffer must be 25 mM, “1×”.
b. Determine the concentration of the stock plasmid solution.
c. In a microcentrifuge tube, add 304.5+/−5% g of plasmid, 300 μL of the “5×”, 125 mM Acetate buffer, and sufficient water to reach a final volume of 1500 μL.
16. Insert a cartridge in to a microfluidic mixing device (e.g., Precision NanoSystems Nano Assembler).
17. Fill a 3 mL BD syringe with the “1×” 25 mM Acetate Buffer and insert it into the left, “aqueous”, channel in the cartridge.
18. Fill a 1 mL BD syringe with Ethanol and insert it into the right, “organic”, channel in the cartridge.
19. Prepare the cartridge by performing a rinse according to the following settings:

| | |
|---|---|
| Total Volume | 4.000 mL |
| Total Flow Rate | 15 mL/minute |
| Left Syringe | 3 mL |
| Right Syringe | 1 mL |
| Start Waste | 0.25 mL |
| End Waste | 0.05 mL |

20. Draw the plasmid solution into a 3 mL BD syringe and insert it into the left, “aqueous”, channel in the cartridge.
21. Draw the lipid solution into a 1 mL BD syringe and insert it into the right, “organic”, channel in the cartridge.
22. Produce a 1500 μL sample according to the following settings:

| | |
|---|---|
| Total Volume | 1.500 mL |
| Total Flow Rate | 15 mL/minute |
| Left Syringe | 3 mL |
| Right Syringe | 1 mL |
| Start Waste | 0.25 mL |
| End Waste | 0.05 mL |

23. Collect the sample in a microcentrifuge tube and gently vortex for 15 seconds.
24. Add 26 μL of the 1% TEMED solution.
25 Add 10.12 μL of the 0.2% HEM solution.
26. Vortex the solution gently for 15 seconds.
27. Place the sample under UV light for 90 seconds.
28. Vortex the solution gently for 15 seconds.
29. Add 10 mL of 1×PBS to a 50 kDa Amicon-Ultra 15 centrifuge filter.
30. Add the SNIPR solution to the centrifuge filter.
31. Centrifuge the sample at 1000 rcf for 30 minutes.
32. Dilute the retentate to 2 mL in 1×PBS, and load onto IZON qEV2 35 nm SEC column.
33. Eluting the sample with 1×PBS, collect and discard the first 14 mL fraction.
36. Collect the next 15 mL fraction and sterilize by filtration through a 45 m PES syringe filter.
35. Add the SNIPR solution to a 50 kDa Amicon-Ultra 15 centrifuge filter.
36. Centrifuge the sample at 1000 rcf for 30 minutes and collect the retentate.

### Results

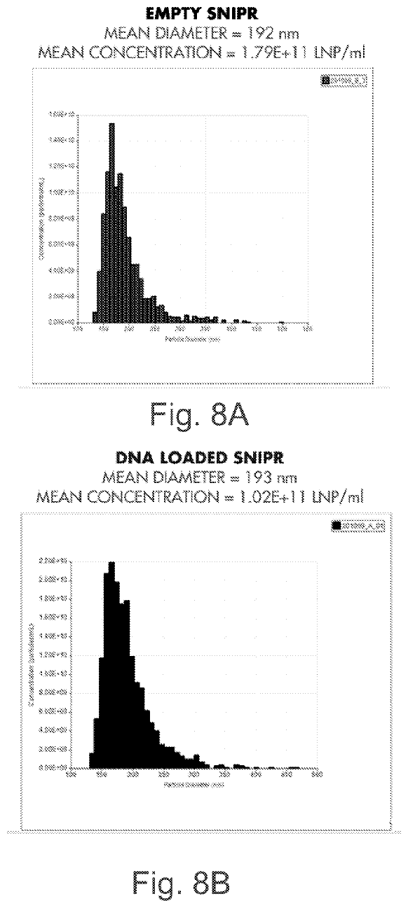

FIGS. 8A and 8B provide plots of hydrodynamic diameter for SNIPR [EMPTY] and SNIPR [pDNA] nanoparticles. The SNIPR manufacturing protocol produces SNIPR nanoparticles with a mean hydrodynamic diameter of approximately 190 nm and low polydispersity. The standard SNIPR manufacturing protocol was used to make a batch of empty SNIPR (SNIPR [EMPTY]) nanoparticles and a batch of SNIPR nanoparticles containing plasmid DNA (SNIPR [pDNA]). The hydrodynamic size and polydispersity of the manufactured SNIPR nanoparticles were quantified by tunable resistive pulse sensing (TRPS) using a qNano Gold nanoparticle analyzer (Izon Science, New Zealand). FIG. 8A provides an analysis of SNIPR [EMPTY] nanoparticles manufactured without encapsulation of a payload. This figure reveals a strong signal indicating that they had a mean diameter of 192 nm. The concentration of the SNIPR [EMPTY] nanoparticles produced was 1.79×10¹¹ LNP/ml. FIG. 8B provides an analysis of SNIPR [pDNA] nanoparticles. This figure deomonstrates that their size (mean diameter of 193 nm) and polydispersity were virtually identical to the SNIPR [EMPTY] nanoparticles referenced above, though their final concentration was somewhat lower (1.02×10¹¹ LNP/ml).

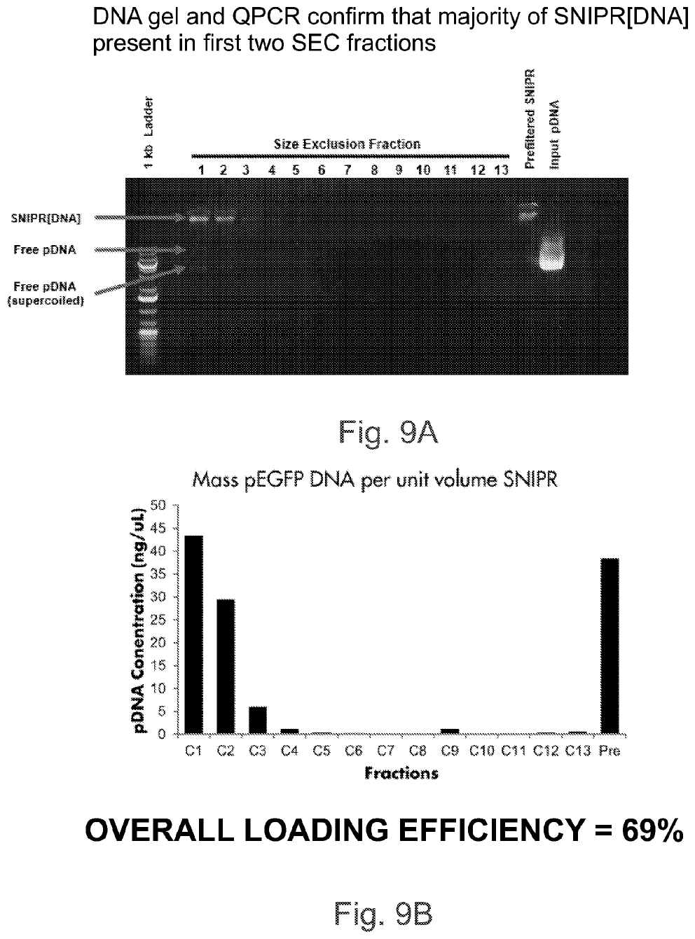

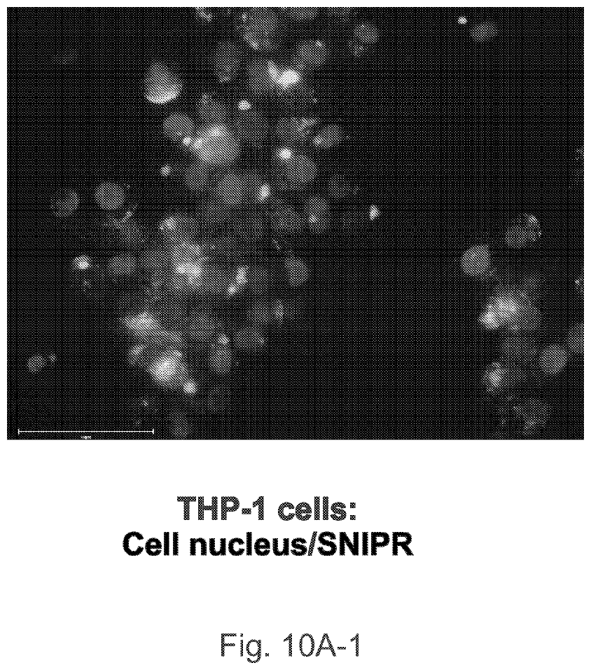

FIGS. 9A and 9B provide visualization and quantitation of pDNA encapsulation SNIPR [pDNA] nanoparticles. FIG. 9A shows the standard SNIPR manufacturing protocol used to make a batch of SNIPR [pDNA] nanoparticles containing plasmid DNA encoding EGFP (pEGFP). An aliquot of the product was taken after SNIPR [pDNA] stabilization by UV crosslinking but before running the product through the SEC columns (“Pre” or “Prefiltered SNIPR”). The remaining product was run through the SEC column, and an aliquot taken from each elution fraction (1-13). The aliquots were visualized via gel electrophoresis (1% agarose gel containing SYBR Safe at the manufacturer's recommended concentration). Plasmid DNA encapsulated within SNIPR is entrapped in the loading wells (arrow labeled SNIPR [pDNA]), with the majority of the visualized plasmid trapped in the wells loaded with elution fractions 1 and 2, and a small amount in the well loaded with elution fraction 3. Very little unencapsulated pDNA is visible in any elution fraction, the majority being in elution fraction 1 (arrows labeled “free pDNA” and “free pDNA (supercoiled)”), indicating very high encapsulation efficiency of pDNA within SNIPR lipid nanoparticles. Lane Key: Lane 1=1 kb size ladder, Lane 2=not loaded, Lanes 3-15=elution fractions 1-13, respectively, Lane 16=prefiltered SNIPR, Lane 17=input DNA (pEGFP from an aliquot of the aqueous input fraction used to manufacture this SNIPR batch), Lanes 18-20=not loaded. In FIG. 9B, the total pDNA encapsulated within SNIPR [pDNA] was calculated by QPCR. An aliquot of the product was taken after SNIPR [pDNA] stabilization by UV crosslinking but before running the product through the SEC columns (“Pre”). The remaining product was run through the SEC column, and an aliquot taken from each elution fraction (1-13). The aliquots were used as templates in a QPCR assay designed to amplify a sequence within the EGFP reporter gene encoded by the SNIPR-encapsulated pEGFP. A serial dilution of the input pEGFP plasmid (without SNIPR encapsulation) was also used as templates in the same QPCR assay to establish a standard curve of pEGFP concentration. All QPCR reactions were carried out on the same plate, under the same reaction conditions, and with the same mixture of primers and QPCR reaction mixture (SYBR Green qPCR, MedChemExpress, Cat no. HY—K0523) to ensure consistency. Plasmid DNA quantity in each elution fraction was calculated from the standard curve using Prism statistical analysis software (GraphPad Software, San Diego, CA). Consistent with the results shown in (A), majority of the detected plasmid was in elution fractions 1 and 2, and a small amount in elution fraction 3. Very little pDNA was detected in any other elution fraction. Key: C1-C13=elution fractions 1-13, respectively, Pre=prefiltered SNIPR.

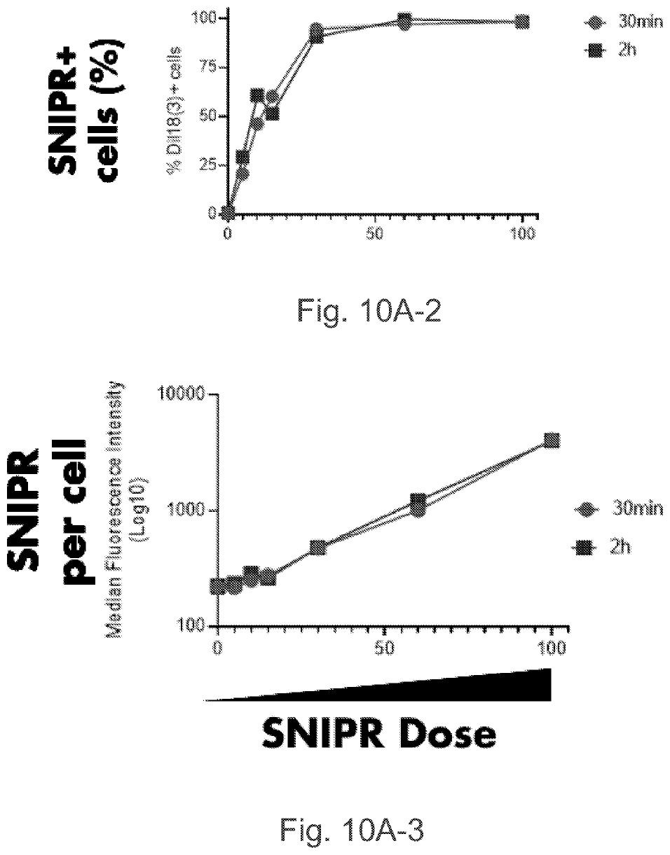

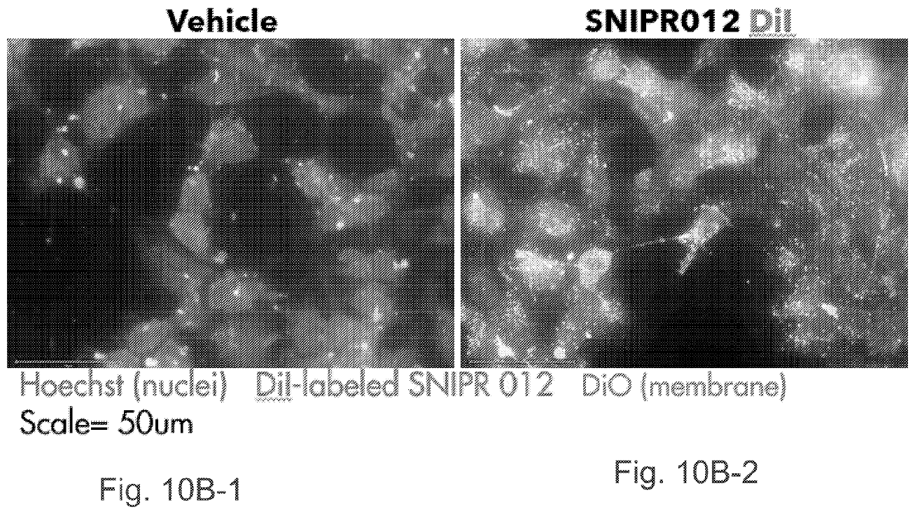

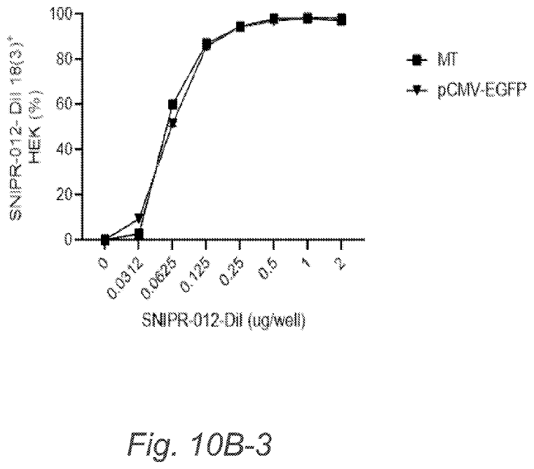

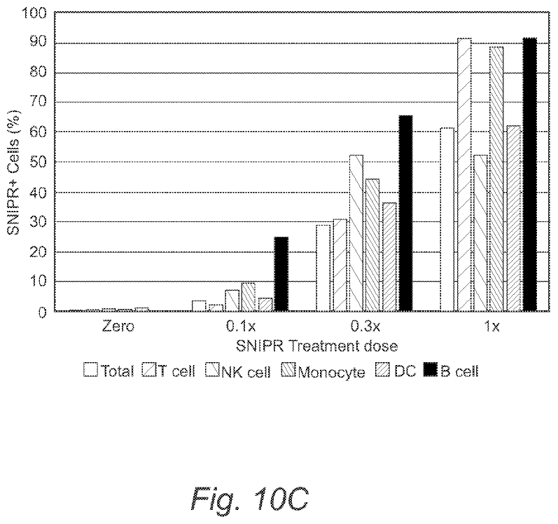

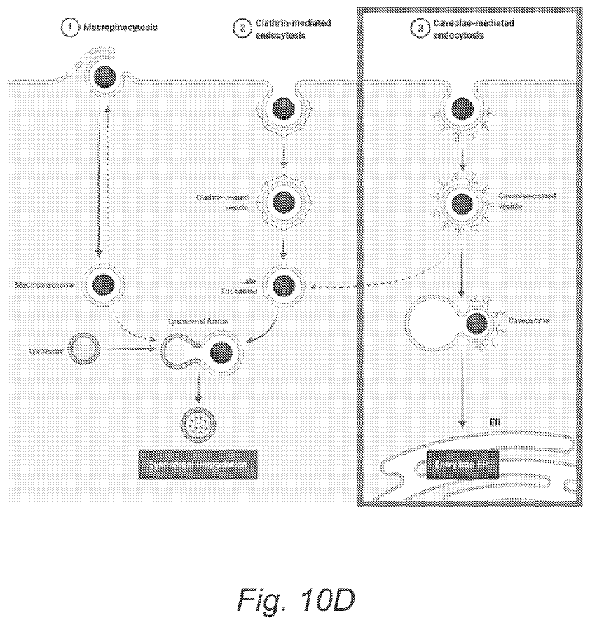

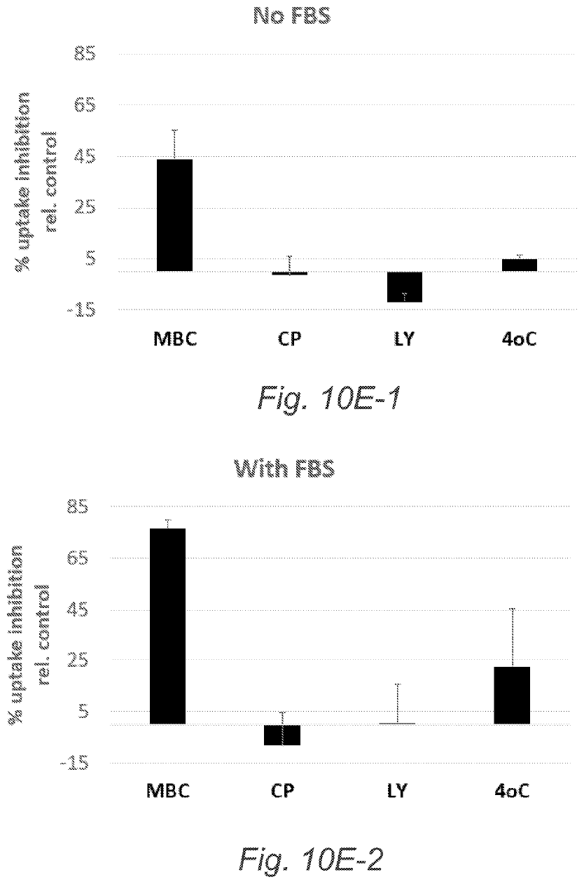

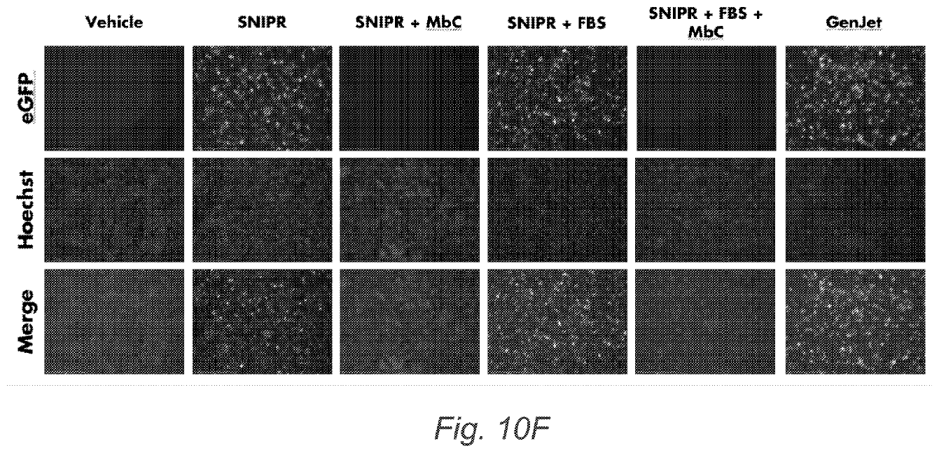

FIGS. 10A-1, 10A-2, 10A-3, 10B-1, 10B-2, 10B-3, 10C, 10D, 10E-1, 10E-2, and 10F: demonstrate that caveolin-mediated uptake is optimal for SNIPR transfection. For FIG. 10A, THP-1 cells were treated with increasing concentration of DiIC18(3)-labeled SNIPR nanoparticles (red) for 30 min and 2 h. Cells were profusely washed and co-stained with nuclear dye (2.5 uM Hoechst-3342, blue). THP-1 cells were imaged and analyzed for uptake efficiency via flow cytometry. For FIG. 10B, HEK-293 cells were treated with fluorescently-labeled SNIPR-012 carrying DNA or fluorescently-labeled empty SNIPR-012. Twenty-four hours post-treatment cells were collected and assessed for uptake as measured by proportion of cells positive for the fluorescent dye (DiI) quantitated via flow cytometry. For FIG. 10C, total primary human peripheral blood mononuclear monocytes (hPBMC) were treated with DiIC18(3)-labeled SNIPR, labeled with antibodies to detect NK cells, T cells, Monocytes, dendritic cells and B cells, and analyzed for uptake via flow cytometry. FIG. 10D shows that there are three main endocytic pathways for uptake of matter smaller than 0.8 uM: 1) Receptor-independent pathway/pinocytosis, 2) Clathrin-mediated endocytosis, and 3) Caveolin-mediated endocytosis. Caveolin-mediated uptake of SNIPR nanoparticles will decrease their chances of lysosomal degradation, thereby increasing the probability of endosomal fusion and successful transfection. It should also be appreciated that these particles undergo LDLR uptake via APOE. For FIG. 10E, Jurkat cells were treated with pathway-specific endocytosis inhibitors for 1 h, namely, methyl-beta-cyclodextrin (MbC, 15 uM), Chloropromazine (CP, 30 uM), or LY-294002 (LY, 20 uM). The pretreated Jurkat cells were then incubated with DiIC18(3)-labeled SNIPR prepared in either RPMI media alone or RPMI media supplemented with 10% FBS for 2 h. One group was incubated at 4° C. to inhibit ATP-dependent endocytosis. Cells were fixed and analyzed for uptake via flow cytometry (n=3). For FIG. 10F, HepG2 cells were pre-treated for 1 h with caveolin inhibitor (15 uM methyl-beta-cyclodextrin, MbC) or vehicle and incubated with vehicle control, SNIPR [pEGFP](SNIPR) or GenJet transfection agent containing 0.5 ug of pEGFP (GenJet) for 48 h. SNIPR [pEGFP] was either prepared in media without FBS or in media supplemented with 10% FBS and incubated for 1 h at 37° C. Cells were stained with nuclear dye (2.5 uM Hoechst-3342, blue) and imaged for EGFP (green) expression analysis.

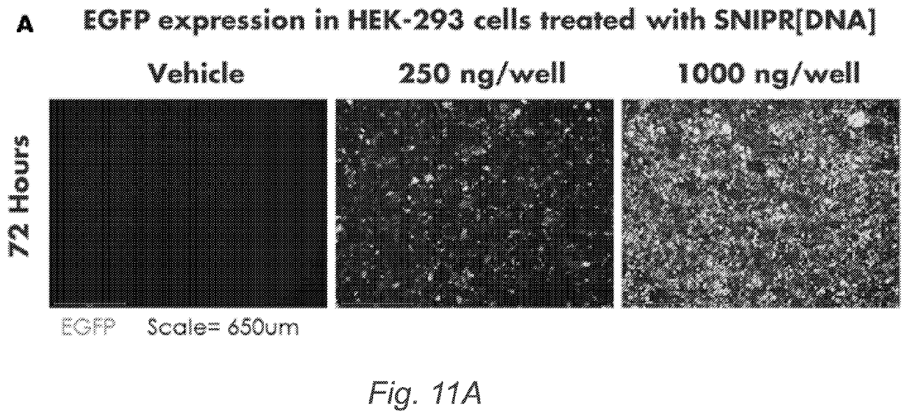

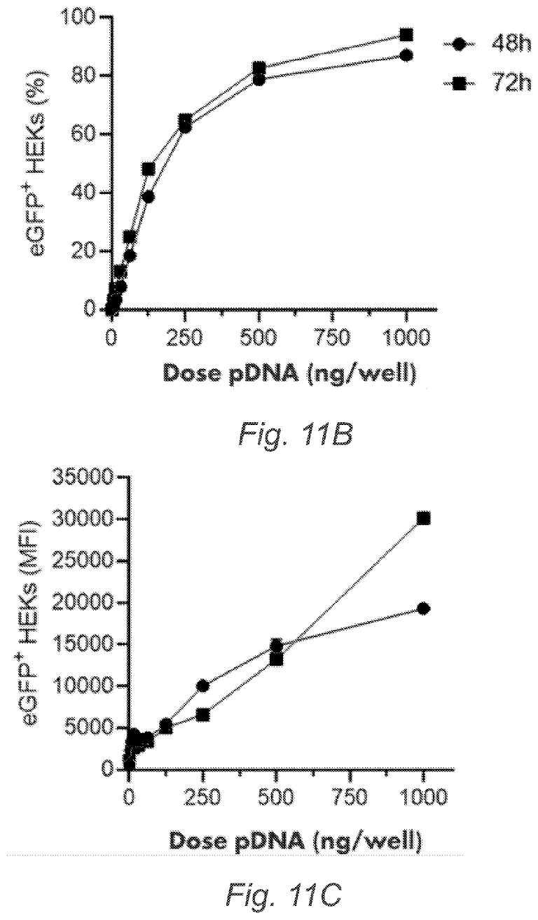

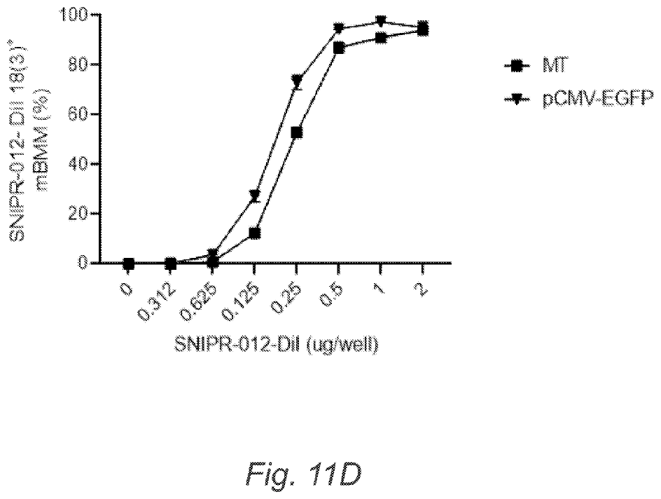

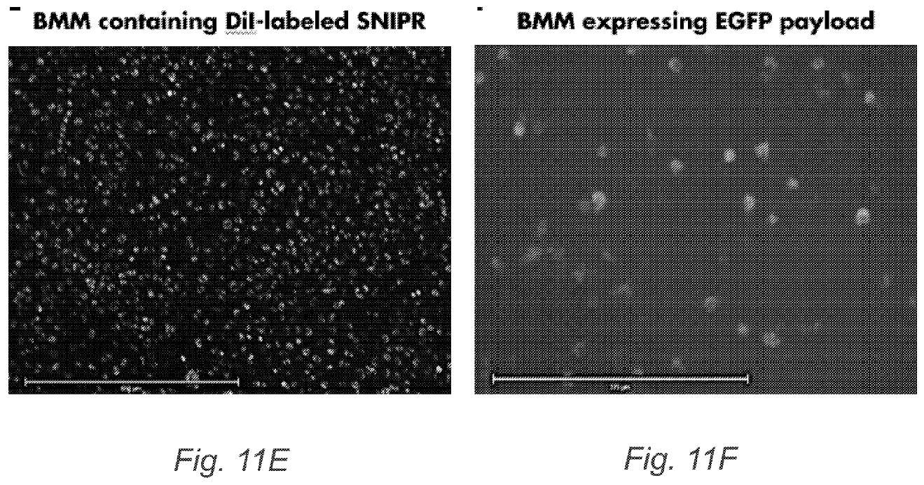

FIGS. 11A, 11B, 11C, 11D, 11E, and 11F demonstrate that SNIPR nanoparticles successfully transfect multiple cell types by delivering plasmid DNA. SNIPR-012 lipid nanoparticles encapsulating plasmid DNA (SNIPR[DNA]) encoding EGFP are efficiently taken up by and mediate transfection of HEK293 cells. HEK-293 were treated with SNIPR[DNA] containing plasmid DNA encoding EGFP or with negative controls. For 11A, EGFP expression was imaged via fluorescence microscopy at 72 hours and 48 hours (data not shown). In addition, cells were collected at 48 hours and 72 hours and transfection efficiency assessed by quantitating the proportion of cells expressing EGFP (FIG. 11B) and their mean fluorescence intensity (MFI, FIG. 11C) via flow cytometry. FIG. 11D shows that SNIPR lipid nanoparticles encapsulating plasmid DNA (SNIPR[DNA]) encoding EGFP and a fluorescent dye (DiI) are efficiently taken up by and mediate transfection of murine bone marrow-derived macrophages (BMMs). BMMs were treated in vitro with fluorescently labeled SNIPR-012 containing pEGFP (blue triangles, pCMV-EGFP) or with equivalent doses of empty SNIPR nanoparticles (red squares, MT) for 24 h. Cells were collected and analyzed for the intracellular presence of the fluorescent dye DiI 18(3) tagged to the LNP. With respect to FIG. 11E, the image represents BMM containing DiI-labeled SNIPR, which was quantitated in the graph. FIG. 11F provides fluorescence microscopy revealing that six days after treatment, the hard-to-transfect BMMs express the EGFP payload.

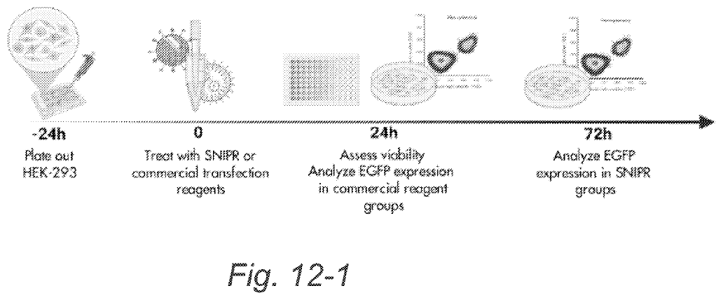

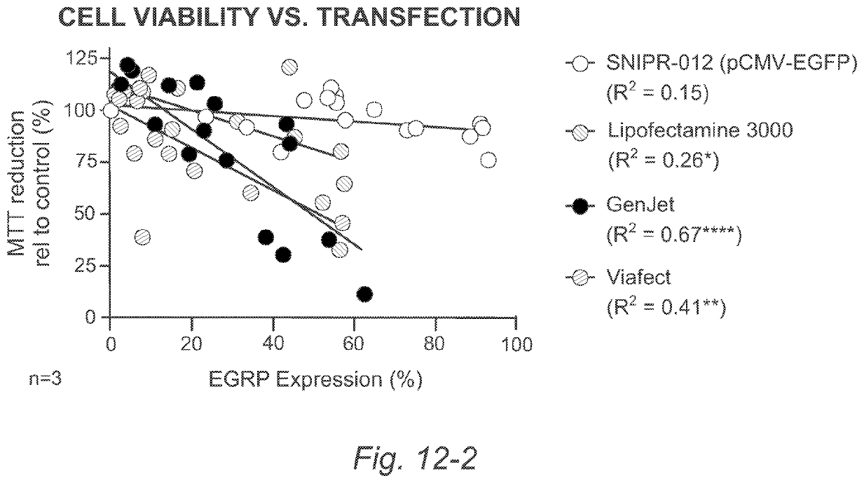

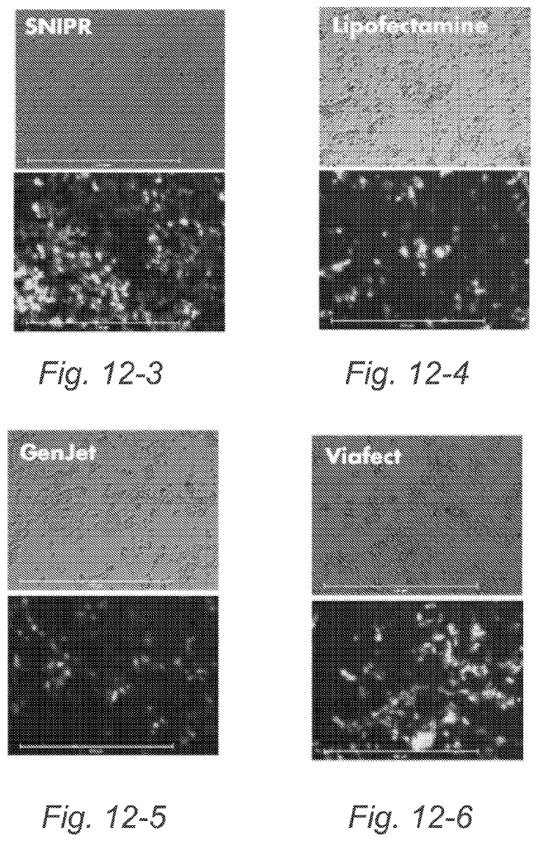

FIGS. 12-1, 12-2, 12-3, 12-4, 12-5, and 12-6 shows that SNIPR lipid nanoparticles encapsulating plasmid DNA mediate highly efficient transfection of cells without the loss of viability observed with off-the-shelf cationic transfection agents. HEK-293 cells were treated with SNIPR-012D, Lipofectamine 3000 (Invitrogen), GenJet (SignaGen) or Viafect (Promega). Six pEGFP concentrations were chosen per group to cover a range of EGFP expression, as directed by the manufacturer's protocol. Twenty-four hours after treatment, cells were collected for viability analysis via MTT reduction assay. Cells were analyzed for transfection efficiency as a proportion of cells EGFP-positive on the peak of expression (i.e. 24 h for cationic transfection reagents and 72 h for SNIPR-012D). Simple linear regression and Pearson's correlation were used to compare groups. Data-points from 3 independent experiments.

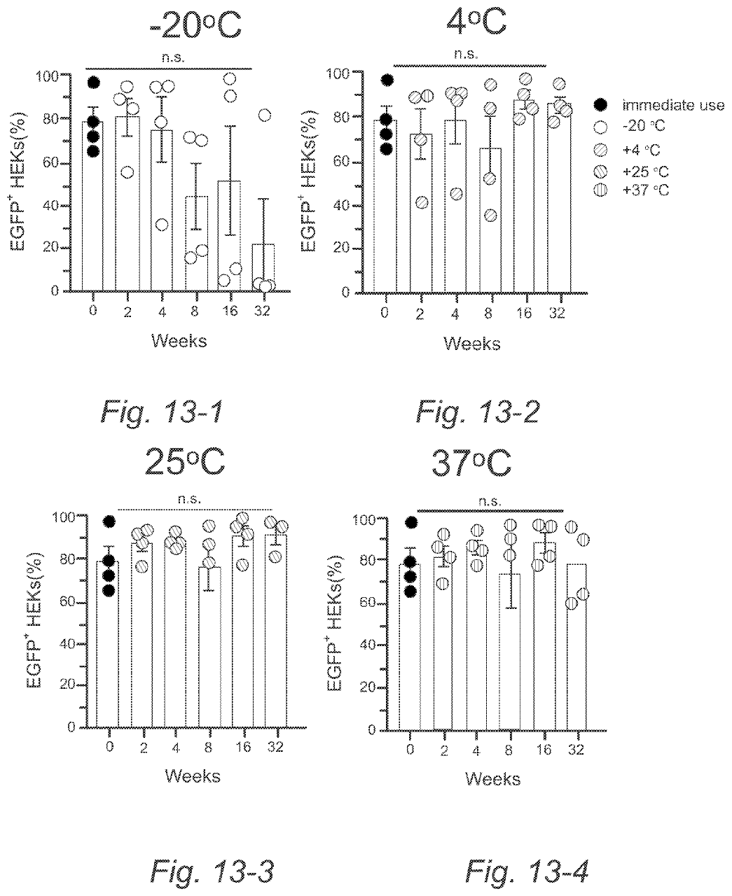

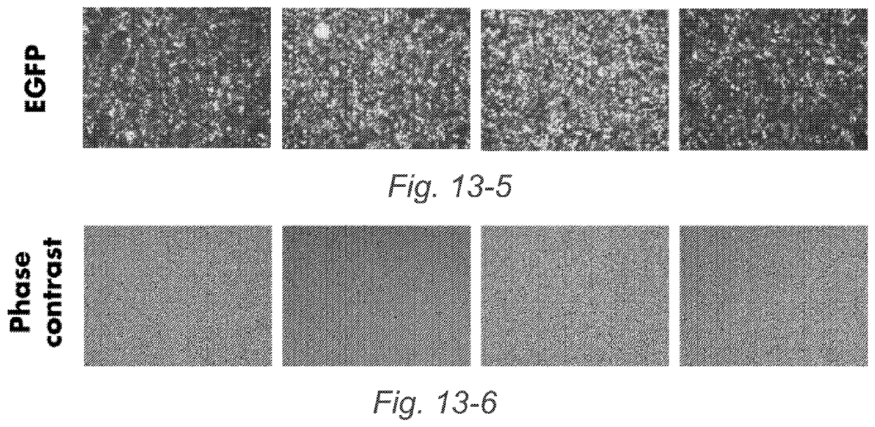

FIGS. 13-1, 13-2, 13-3, 13-4, 13-5, and 13-6 shows that SNIPR lipid nanoparticles encapsulating plasmid DNA are extremely stable and retain the ability to transfect cells even after storage for at least 32 weeks at ambient temperatures of 4 C, 25 C, and 37 C. Four separate batches of SNIPR-12 encapsulating pEGFP plasmid were manufactured and divided into 200 ul aliquots. One aliquot from each batch was stored at −20 C, +4 C, +25 C, and +37 C in sterile PBS for zero (i.e, used immediately) 2, 4, 8, 16, and 32 weeks. No stabilizing excipients were added to the sterile PBS. For each storage time point, HEK293 cells were treated separately with one of the stored aliquots or an equivalent volume of sterile PBS. Sixty-three hours after treatment cell were imaged by fluorescence microscopy to confirm EGFP (green) expression. The fraction of treated cells that were EGFP-positive was quantitated by flow cytometry. Bar charts show mean percentage of EGFP-positive HEK cells per treatment group (n=4 replicates per time point and storage temperature). Error bars indicate SEM. Filled circles indicate mean percentage of EGFP-positive HEK cells for each individual replicate at each time point and storage temperature. Colors of filled circles indicate storage temperature. There was no statistically significant change in the percentage of EGFP+ cells after treatment with SNIPR[pDNA] LNPs stored under any of the experimental conditions. There was a noticeable trend in decreased transfection in cells treated with SNIPR[pDNA] stored at −20 C for longer than 4 weeks.

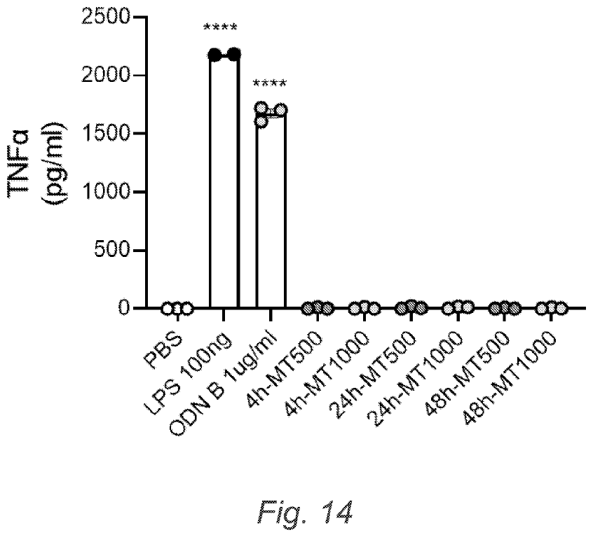

FIG. 14 shows that empty SNIPR nanoparticles elicit no induction of TNFa release by macrophages even with 48 hours of exposure. As expected, the highly immunogenic positive controls lipopolysaccharide (LPS) and CpG oligodeoxynucleotide (ODN B) strongly elicited TNFa release by murine bone marrow-derived macrophages. In contrast, doses of empty SNIPR LNPs equivalent to those required to deliver 500 or 1000 nanograms of DNA (MT500 and MT1000, respectively) elicited no TNFa release from cells exposed to them for 4, 24, or 48 hours. As expected, the negative vehicle control (PBS) also elicited no TNFa release. TNFa release was quantitated by ELISA. Bar charts show mean TNFa release per treatment group (n=3 replicates per treatment group). Error bars indicate SEM. Filled circles indicate mean TNFa release for each individual replicate for each treatment group. Colors of filled circles indicate treatment group.

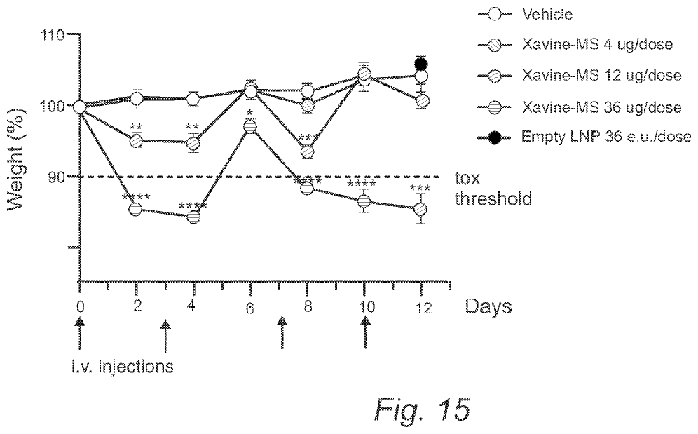

FIG. 15 shows that empty SNIPR nanoparticles elicit no weight loss in mice dosed four times over the course of two weeks. Groups of mice (n=8 per group) were treated four times with MS-Xavine™ LNPs delivering low, medium, and high doses of DNA (4 ug/dose, 12 ug/dose, 36 ug/dose, respectively), or an amount of empty Xavine™ LNPs equal to the number used in the 36 ug/dose group, or vehicle control (sterile saline for injection). The body weights of the mice were monitored over the course of the experiment. Mice in the high dose group showed strongly statistically significant body weight loss compared to vehicle-treated controls (***p<0.001 at final time point). This weight loss was below the 20% body weight loss humane endpoint mandated by our IACUC protocol. Mice in the medium dose group showed moderate weight loss which had resolved by the end of the experiment. In contrast, mice in the low MS-Xavine™ group and in mice treated with empty LNPs never showed any weight loss compared to the vehicle-only controls at any time during the experiment.

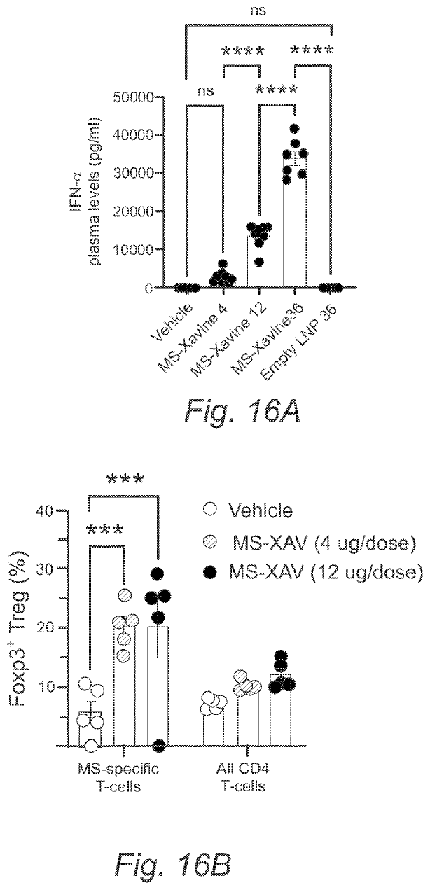

FIGS. 16A and 16B shows that MS-Xavine™ nanoparticles administered at a dose sufficient to elicit an increase in antigen-specific regulatory T cells in vivo induce low IFNa release in mice 6 hours after treatment. Mice were treated four times with MS-Xavine™ LNPs delivering low, medium, and high doses of DNA (4 ug/dose, 12 ug/dose, 36 ug/dose, respectively), or an amount of empty Xavine™ LNPs equal to the number used in the 36 ug/dose group, or vehicle control (sterile saline for injection). For FIG. 16A, plasma samples were collected from each mouse 6 hours after the first dose was administered, and IFNa levels quantitated by ELISA. Mice in the medium and high MS-Xavine™ dose groups showed strongly statistically significant increases in IFNa secretion compared to saline controls and empty LNP controls (indicating that IFNa release was associated with Xavine™ DNA payload rather than the SNIPR LNP delivery system). Mice treated with the low MS-Xavine™ dose also showed an increase in IFNa secretion compared to controls, but this was not statistically significant. For FIG. 16B, mice from the vehicle control, low, and medium MS-Xavine™ dose groups were euthanized, and spleens harvested. Splenocytes were analyzed by flow cytometry. Splenocytes of mice in both the low and medium dose groups showed strongly statistically significant increases in MS-specific (MOG tetramer positive) CD4 T cells that were FOXP3+ regulatory T cells (Tregs) compared to the splenocytes of the vehicle control-treated mice.

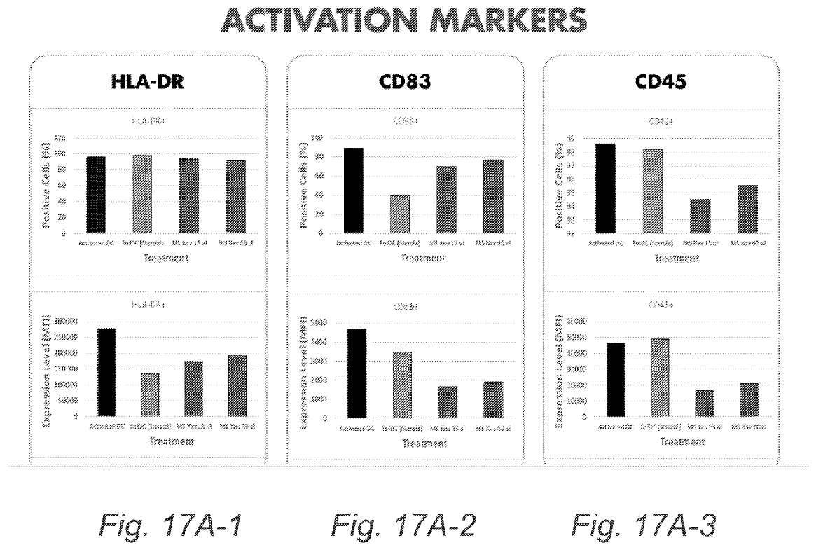

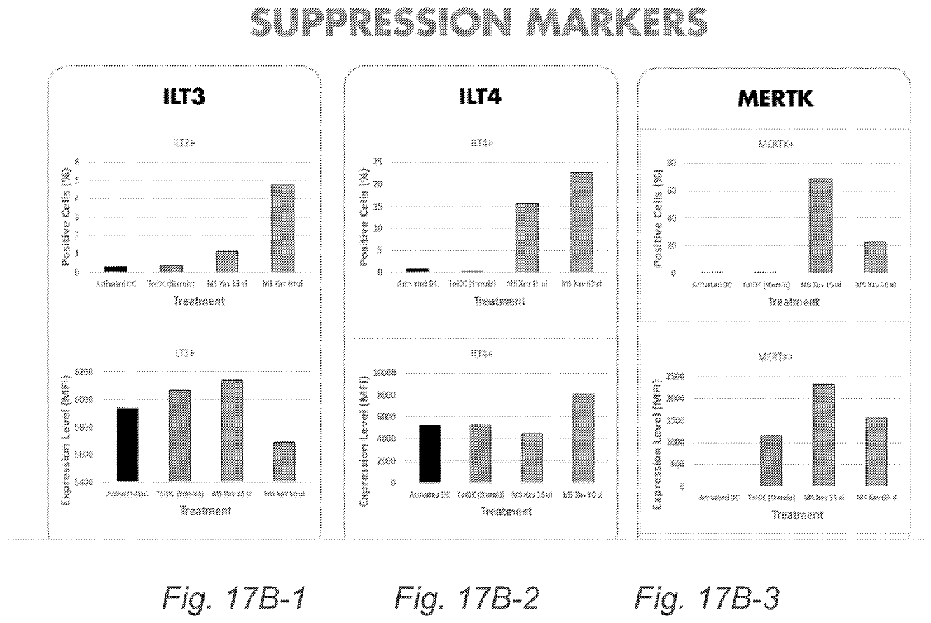

FIGS. 17A-1, 17A-2, 17A-3, 17B-1, 17B-2, and 17B-3 show that prototype MS-Xavine™ nanoparticles induce tolerogenic antigen-presenting cells (APC). Primary human CD14-derived dendritic cells (DC) were treated with a single dose of MS-Xavine™ for 72 h. Cells were stained with amine-reactive dye-EF506 and fluorescently labeled antibodies against human HLA-DR, CD83, CD45, ILT3, ILT4 and MERTK and analyzed expression of markers via flow cytometry.

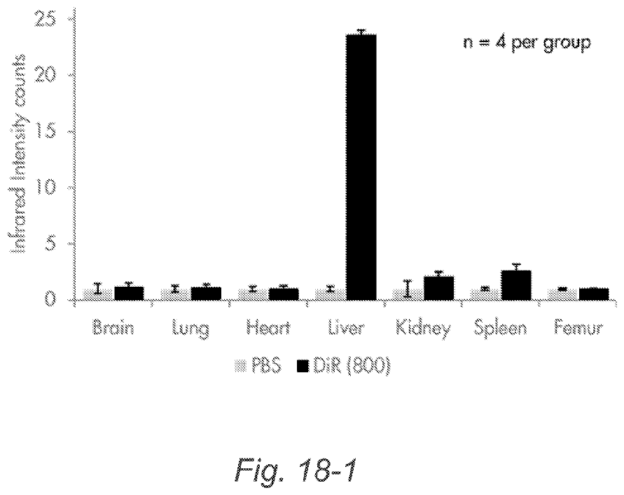

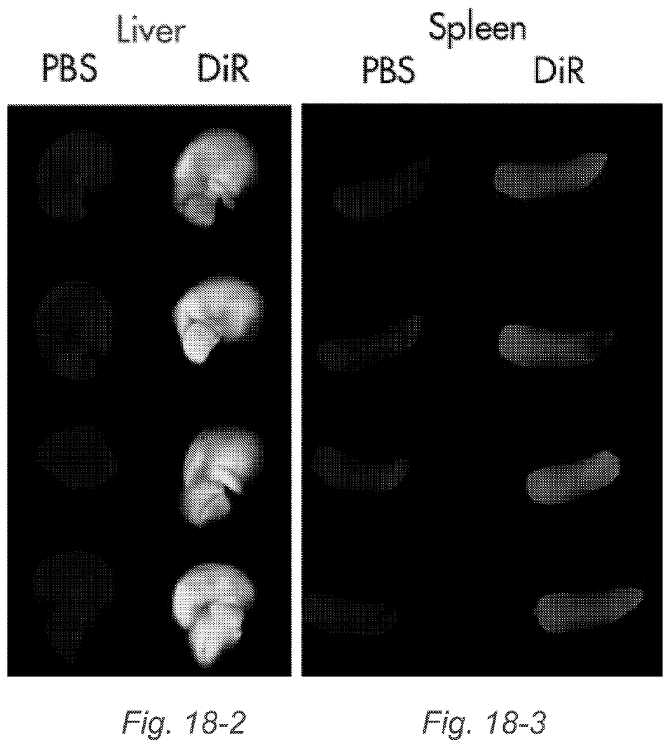

FIGS. 18-1, 18-2, and 18-3 shows that near-infrared dye labeled SNIPR nanoparticles biodistributed primarily to the liver and spleen of mice within 24 hours. Plasmid DNA-loaded SNIPR LNPs were labeled with a near-infrared (nIR) dye (DiR) and administered by tail vein injection to groups of naïve mice (n=4 per group). Each mouse received a single dose of nIR-labeled SNIPR delivering 12 ug plasmid DNA encoding the autoantigen MOG. The mice were euthanized 24 hours after dosing, and their organs harvested and fixed in paraformaldehyde. After fixation, the nIR signal in each organ was quantitated using iBright FL1000 imager system (ThermoFisher Scientific). The administered nIR-labeled SNIPR accumulated primarily in two major lymphoid organs, the liver and spleen.

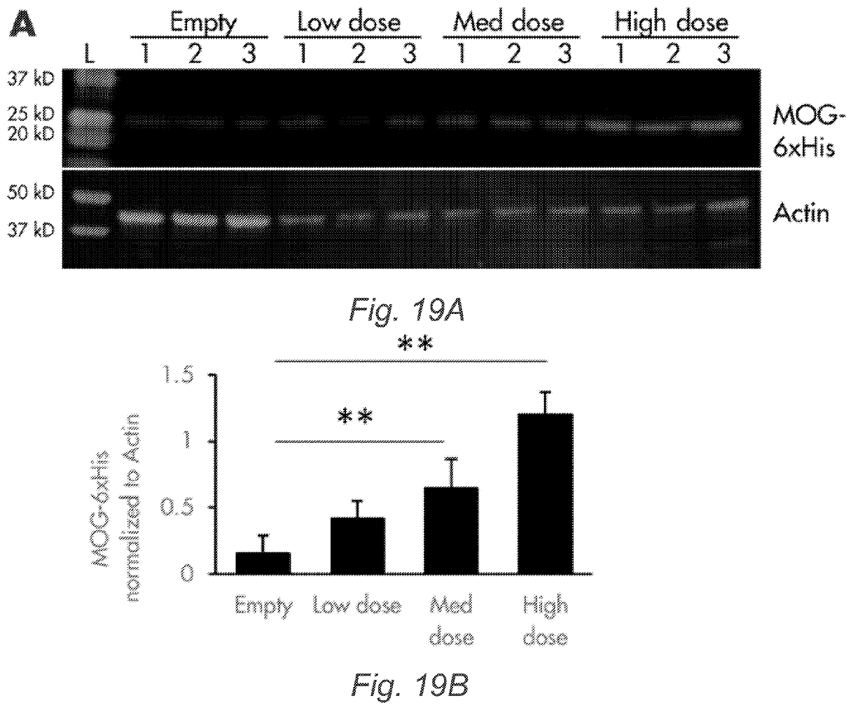

FIGS. 19A and 19B shows that MS-Xavine nanoparticles delivering plasmid DNA encoding the multiple sclerosis autoantigen MOG elicit dose-dependent expression of MOG protein in the spleens of treated mice. A.) Groups of naïve mice (n=5) were treated four times with three doses of plasmid DNA encoding MOG (low=4 ug/dose, med=12 ug/dose, high=36 ug/dose), or empty SNIPR LNPs (equivalent to the 36 ug/dose group). Protein was isolated in triplicate from the membrane fraction of splenocytes harvested from each mouse, and quantitative Western blot carried out. B.) MOG-6×His protein expression was normalized to expression of a housekeeping control (actin). Normalized MOG-6×His expression increased in a MS-Xavine dose-dependent manner and was statistically significantly higher in the medium and high dose groups compared with empty SNIPR controls (**p<0.01).

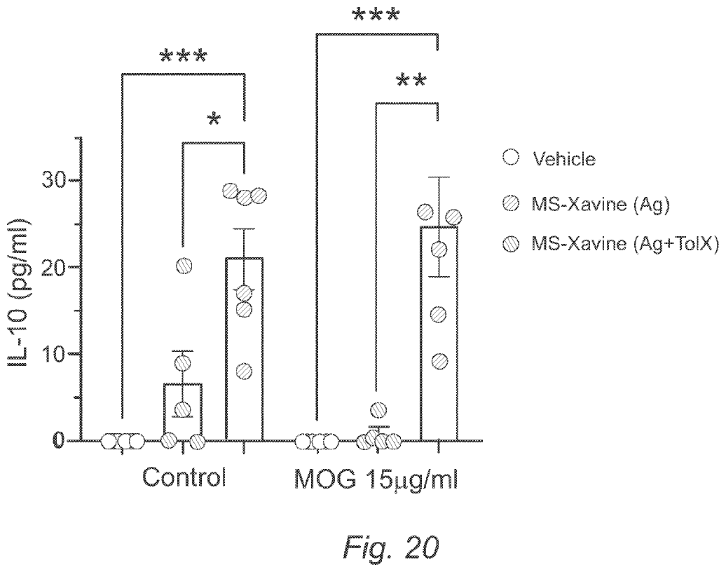

FIG. 20 shows that IL-10 secretion is strongly upregulated in the splenocytes of mice treated with MS-Xavine nanoparticles delivering plasmid DNA encoding the multiple sclerosis autoantigen MOG plus IL-10. A.) Groups of naïve mice (n=5) were treated four times with MS-Xavine containing DNA encoding only MOG (MS-Xavine Ag) or DNA encoding MOG and IL-10 (MS-Xavine Ag+TolX). Scrambled insert plasmid was used to control total amount of DNA administered (12 ug/dose/mouse). Splenocytes isolated from the mice were cultured with and without MOG restimulation, and IL-10 secretion quantitated by ELISA. Mice treated with the MS-Xavine Ag+Tol variant secreted statistically significantly more IL-10 than the negative control mice treated with sterile PBS for injection, regardless of MOG restimulation (***p<0.001 with MOG restimulation, **p<0.01 without). This indicates that MS-Xavine nanoparticles successfully deliver IL-10 plasmids to splenocytes in treated animals, and that proper expression, translation, and secretion of IL-10 occur.

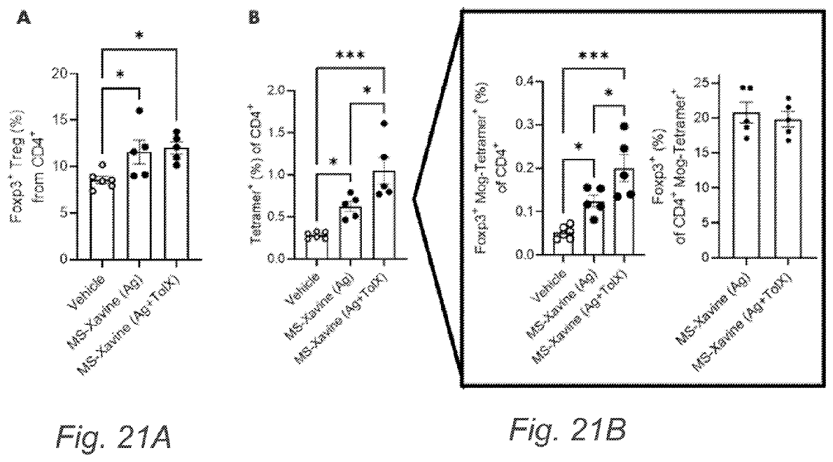

FIG. 21 shows that addition of a tolerizing factor (IL-10 plasmid) to MS-Xavine increases the total number of antigen-specific Tregs, but not their proportion within the antigen-specific CD4+ T cell population. A.) Splenocytes isolated from the same mice described above (FIG. 20) for analysis by flow cytometry. Bar charts show mean percentage of CD4+ T cells that were positive for the Treg-specific marker FOXP3 in each treatment group (n=5 or 6 animals per treatment group). Error bars indicate SEM. Filled circles indicate mean percentage of CD4+ T cells that were positive for the Treg-specific marker FOXP3 in each individual animal. Colors of filled circles indicate treatment group (white=saline vehicle control; purple=MS-Xavine (Ag-only variant); green=MS-Xavine (Ag+TolX variant)). Both MS-Xavine variants elicited a statistically significant increase in splenic CD4+FOXP3+ Tregs (*p<0.05). B.) Mice treated with the Ag+TolX variant showed a statistically significant increase in MOG-specific CD4+FOXP3+ Tregs compared to both the vehicle control and the Ag-only variant. Interestingly, this was in the context of a general increase in MOG-specific CD4+ T cells; the ratio of MOG-specific CD4+FOXP3+ Tregs was approximately 20% in mice treated with either MS-Xavine. Bar charts show mean percentage of T cells that were positive for the markers indicated in the y-axis legend of each chart in each treatment group (n=5 or 6 animals per treatment group). Error bars indicate SEM. Filled circles indicate mean percentage of T cells that were positive for the markers indicated in the y-axis legend of each chart in each individual animal.

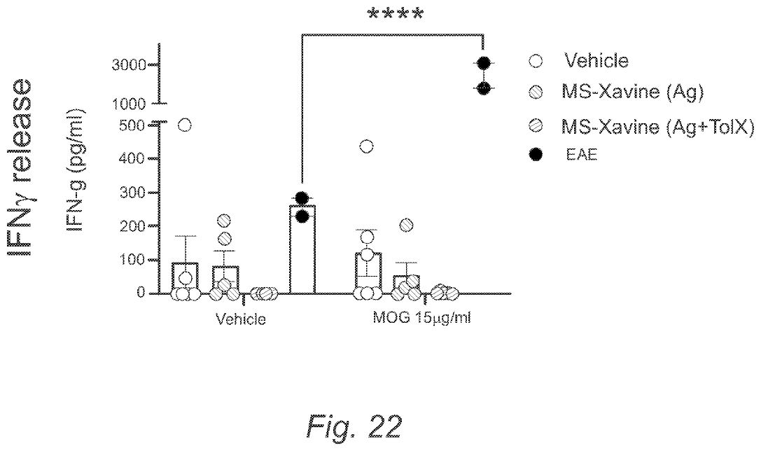

FIG. 22 shows that restimulation with MOG does not induce IFNg secretion by MOG-reactive splenocytes isolated from mice treated with either MS-Xavine variant, and the addition of the IL-10 tolerizing factor completely abrogates IFNg secretion. Splenocytes isolated from the same mice described above (FIG. 20) were cultured and restimulated with MOG peptide or vehicle control. (A) As expected, cells isolated from the spleens of MOG-induced experimental autoimmune encephalitis (EAE) positive control mice strongly upregulated IFNg expression upon restimulation with MOG (****p<0.0001). Splenocytes of mice treated with the Ag-only MS-Xavine variant showed no change in IFNg secretion in response to MOG restimulation, suggesting that the increased MOG-specific CD4+ cells observed in these mice are not pro-inflammatory Tresp cells. Interestingly, splenocytes of mice treated with the Ag+Tol MS-Xavine showed no IFNg secretion, suggesting exposure to an immunosuppressive microenvironment.

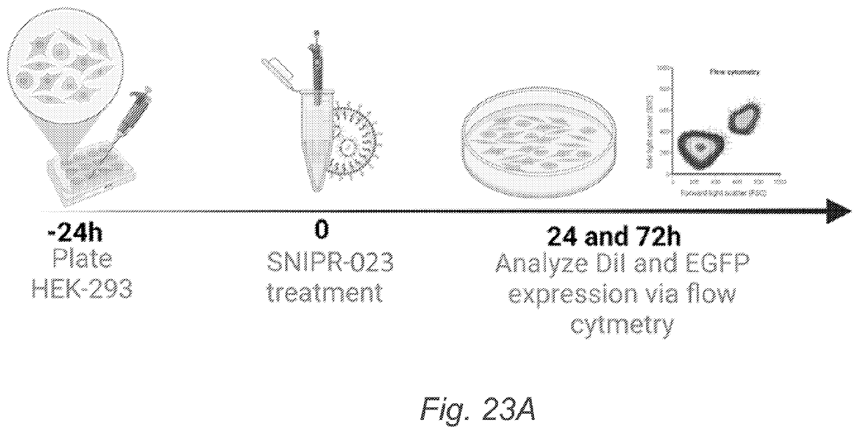

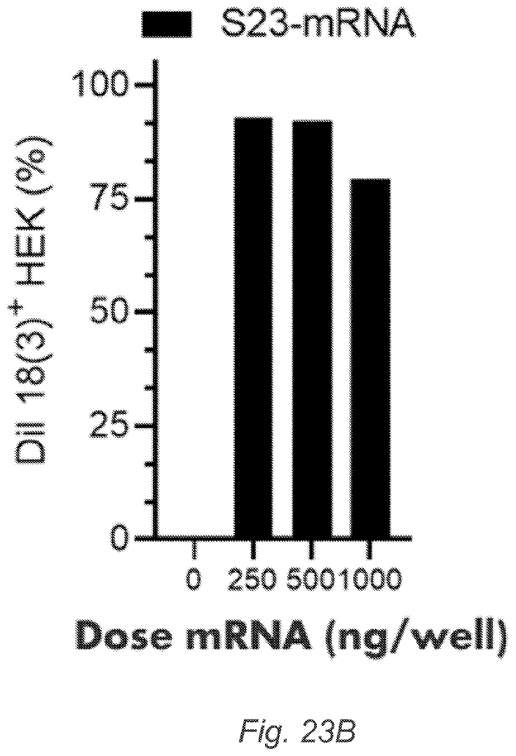

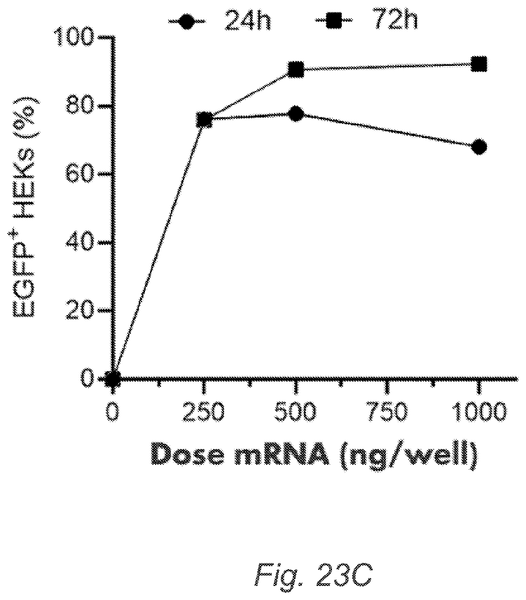

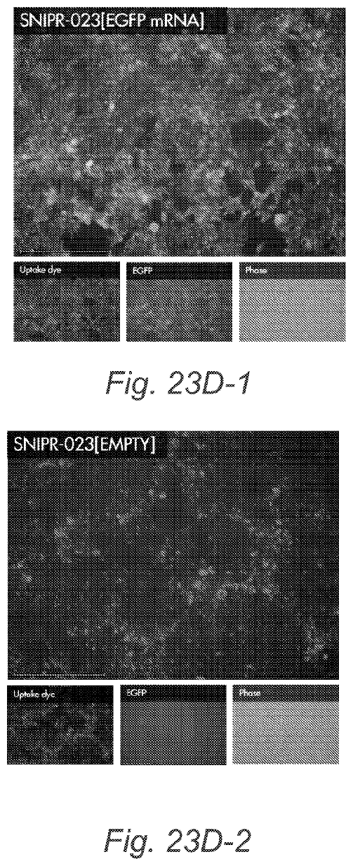

FIGS. 23A, 23B, 23C, 23D-1, and 23D-2 shows that SNIPR-023 lipid nanoparticles encapsulating messenger RNA encoding EGFP and labeled with a fluorescent dye (DiI) are efficiently taken up by and mediate transfection of HEK293 cells. For FIG. 23A, HEK-293 were treated with DiI labeled SNIPR-023 containing mRNA encoding EGFP (SNIPR-023[mRNA]) or empty SNIPR LNPs (SNIPR-23[Empty]) as controls. For FIG. 23B, twenty-four hours after treatment cells were assessed for LNP uptake via flow cytometry by quantifying the proportion of cells positive for the fluorescent label DiI. For FIG. 23C, HEK-293 cells were also assessed for transfection efficiency by quantitating the proportion of cells expressing EGFP via flow cytometry. Seventy-two hours after treatment, cells were collected and assessed for EGFP expression via flow cytometry. FIGS. 23D-1 and 23D-2 provide representative fluorescence microscopy showing uptake of both SNIPR[mRNA] and SNIPR[Empty](red spots) and EGFP expression only in cells treated with SNIPR[mRNA].

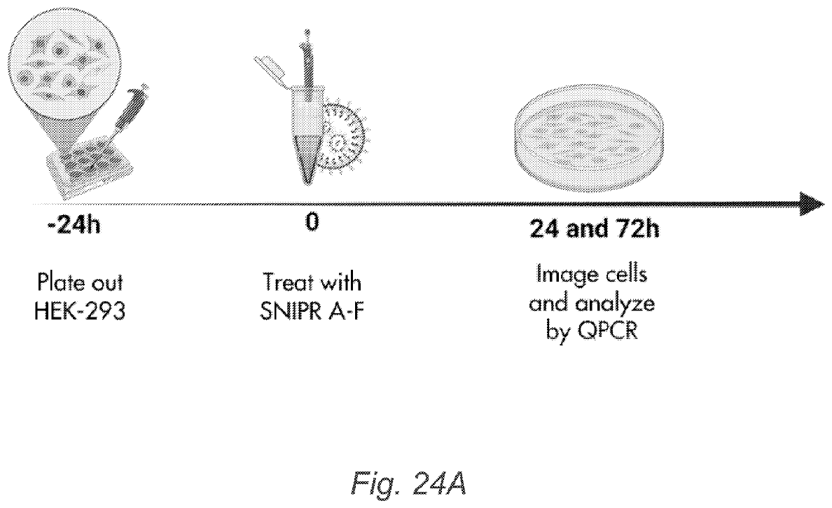

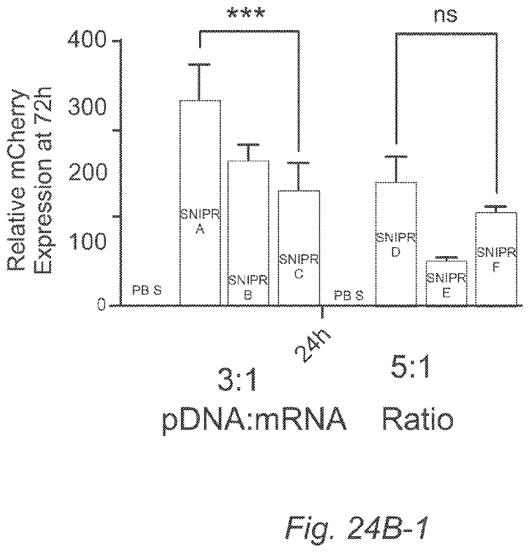

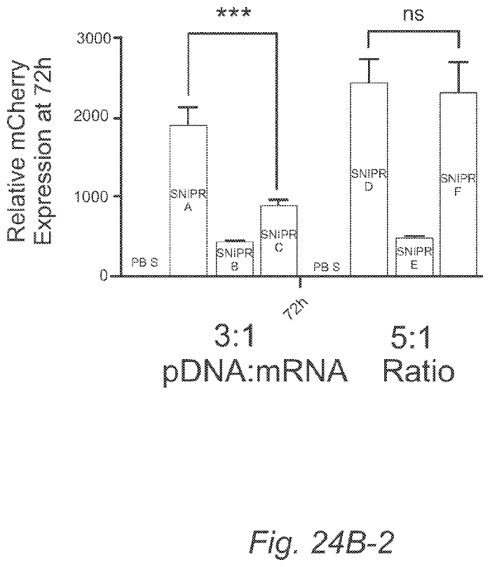

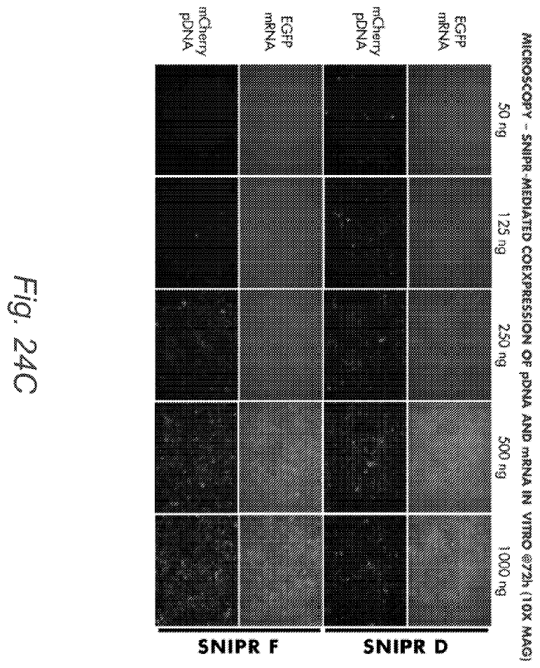

FIGS. 24A, 24B-1, 24B-2, and 24C shows that various SNIPR lipid nanoparticles co-encapsulating plasmid DNA encoding mCherry and RNA encoding EGFP are efficiently taken up by and mediate dual transfection of HEK293 cells. For FIG. 24A, HEK-293 cells were treated with various doses of six different SNIPR[pDNA+mRNA] formulations (SNIPR A-F) containing pDNA and mRNA encoding EGFP encoding mCherry at either a 5:1 pDNA:mRNA ratio (SNIPR A-C) or a 3:1 pDNA:mRNA ratio (SNIPR D-F). SNIPR A-F formulations also differed by the ratio of two different ionizable lipids included in the formulations, with the following pairs of formulations containing the same ratios of ionizable lipids: SNIPR A and D, SNIPR B and E, SNIPR C and F. For FIG. 24B-1, HEK-293 cells were assessed for transfection efficiency by quantitating the expression of EGFP mRNA (at 24 hours post-treatment) via quantitative PCR. For FIG. 24B-2, HEK-293 cells were assessed for transfection efficiency by quantitating the expression of mCherry pDNA (72 hours post-treatment) via quantitative PCR. Both the ratio of pDNA:mRNA and the ratio of ionizable lipids contained in the various formulations affected transfection efficiency of pDNA and mRNA. FIG. 24C provides representative fluorescence microscopy showing dose-responsive simultaneous dual transfection of both mCherry pDNA and EGFP mRNA in cells treated with the SNIPR D and SNIPR F formulations.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

Sankaram, Mantripagada B., and Sinil Kim. Preparation of Multivesicular Liposomes for Controlled Release of Active Agents. Mantripragada B. Sankaram, assignee. Patent CA2199004 C. 13 Sep. 1994.
Kim, Sinil, and Stephen B. Howell. Multivesicular liposomes with controlled release of active agents encapsulated in the presence of a hydrochloride. Sinil Kim, assignee. Patent U.S. Pat. No. 6,071,534 A. 15 Feb. 1988.
Rovira-Bru, M., Thompson, D. H., Szleifer, I. “Size and Structure of Spontaneously Forming Liposomes in Lipid/PEG-Lipid Mixtures.” Biophysical Journal. 2002. 83(5); 2419-2439.
Gillespie D T. Exact stochastic simulation of coupled chemical reactions. J Phys Chem. 1977; 81(25):2340-2361. doi:10.1021/j100540a008.
Alberts B, Johnson A, Lewis J, et al. Molecular Biology of the Cell. 4th edition. New York: Garland Science; 2002. The Lipid Bilayer. Available from: https://www.ncbi.nlm.nih.gov/books/NBK26871/.

1-50. (canceled)

51. A method of forming a multivesicular lipid composition including a plurality of multivesicular lipids, each multivesicular lipid nanoparticle comprising:
a carrier phospholipid micelle including a carrier phospholipid layer comprising carrier phospholipids and having an average diameter less than 1 micron; and
at least one sub-chamber phospholipid inverse micelle including a sub-chamber phospholipid layer comprising sub-chamber phospholipids and having an average diameter less than 100 nm, the carrier phospholipid micelle encapsulating the at least one sub-chamber phospholipid inverse micelle, the method comprising:
a) combining an organic solvent in which carrier phospholipids and sub-chamber phospholipids are dissolved with an aqueous solvent in which a hydrophilic therapeutic/diagnostic payload is dissolved; and
b) forming a plurality of sub-chamber phospholipid inverse micelles, the plurality of sub-chamber phospholipid inverse micelles having an average nanoparticle diameter less than 100 nm, the plurality of sub-chamber phospholipid inverse micelles being formed from a first MVL-forming composition that includes the hydrophilic therapeutic/diagnostic payload; and
c) forming a plurality of carrier phospholipid micelles having an average nanoparticle diameter less than 1 micron, the plurality of carrier phospholipid micelles being formed from second MVL-forming composition that includes the plurality of sub-chamber phospholipid inverse micelles such that sub-chamber phospholipid inverse micelles are encapsulated by carrier phospholipid micelles.
d) covalently linking phospholipid tail groups of the plurality of carrier phospholipid micelles and the plurality of sub-chamber phospholipid inverse micelles via a chemical reaction catalyzed by application of actinic radiation.

52. The method of claim 51, wherein the first MVL-forming composition includes 1,2-dipalmitoleoyl-sn-glycero-3-phosphocholine or residues thereof, 1,2-dipalmitoyl-sn-glycero-3-[phospho-rac-(3-lysyl(1-glycerol))](chloride salt) or residues thereof, 1-palmitoyl-2-[16-(acryloyloxy)palmitoyl]-sn-glycero-3-phosphorylcholine or residues thereof, 1,2-dipalmitoyl-sn-glycero-3-phosphoethanolamine-N-[methoxy(polyethyleneglycol)-2000](ammonium salt) or residues thereof; 2,2-dilinoleyl-4-dimethylaminoethyl-[1,3]-dioxolane or residues thereof; and Cholest-5-en-30-ol or residues thereof.

53. The method of claim 51 wherein an aqueous fraction of the first MVL-forming composition includes one or more additional hydrophilic therapeutic/diagnostic payloads.

54. The method of claim 51 wherein an organic fraction of the first MVL-forming composition includes one or more hydrophobic therapeutic/diagnostic payload(s).

55. The method of claim 53 wherein an organic fraction of the first MVL-forming composition includes one or more hydrophobic therapeutic/diagnostic payload(s).

56. A multivesicular lipid nanoparticle composition including a plurality of multivesicular lipid nanoparticles, each multivesicular lipid nanoparticle comprising:
a carrier lipid nanoparticle including a carrier micelle comprising carrier lipids selected from the group consisting of phospholipids, lipids not including phosphorus, and combinations thereof, the carrier lipid nanoparticle having an average diameter less than 1 micron; and
at least one sub-chamber lipid micelle nanoparticle including a sub-chamber inverse micelle comprising sub-chamber lipids selected from the group consisting of phospholipids, lipids not including phosphorus, and combinations thereof, the at least one sub-chamber lipid micelle nanoparticle having an average diameter less than 100 nm, wherein the carrier lipid nanoparticle encapsulates at least one sub-chamber lipid nanoparticle.

57. The multivesicular lipid nanoparticle composition of claim 56, wherein the carrier micelle and/or the sub-chamber inverse micelle independently include a lipid described by the following formula: 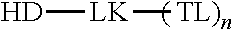
wherein:
HD is a head group;
LK is a linking group;
TL is a tail group; and
n is an integer representing a number of tail groups.

58. The multivesicular lipid nanoparticle composition of claim 57, wherein when there are more than one tail groups, the tail groups can be the same or different.

59. The multivesicular lipid nanoparticle composition of claim 57, wherein the tail group include branched or unbranched C₆₋₂₅ aliphatic chains.

60. The multivesicular lipid nanoparticle composition of claim 59, wherein one or more carbon atoms in the branched or unbranched C₆₋₂₅ aliphatic chains can be replaced with O, N, S, or a carbonyl.

61. The multivesicular lipid nanoparticle composition of claim 59, wherein the branched or unbranched C₆₋₂₅ aliphatic chains can include one or more ester groups.

62. The multivesicular lipid nanoparticle composition of claim 57, wherein the head group includes a moiety selected from the group consisting of:

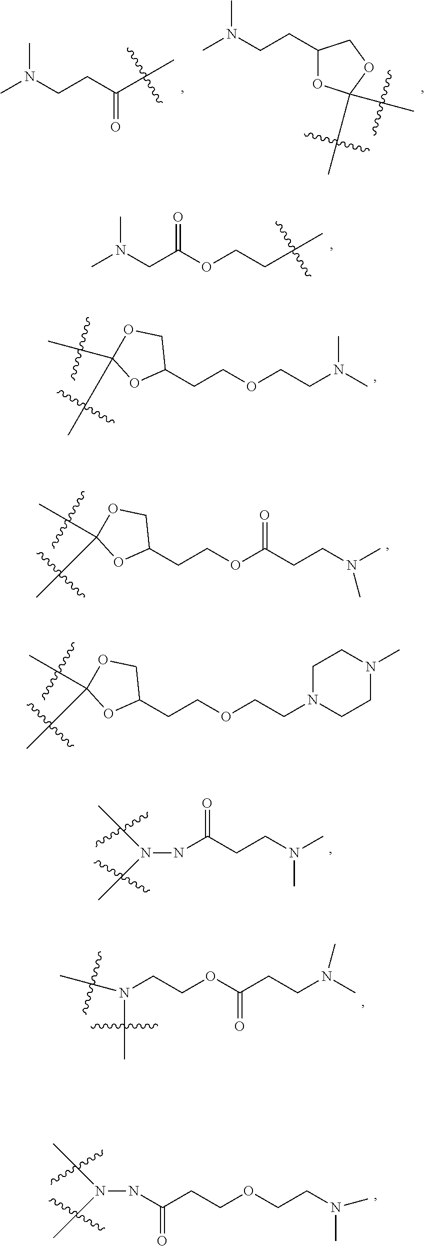

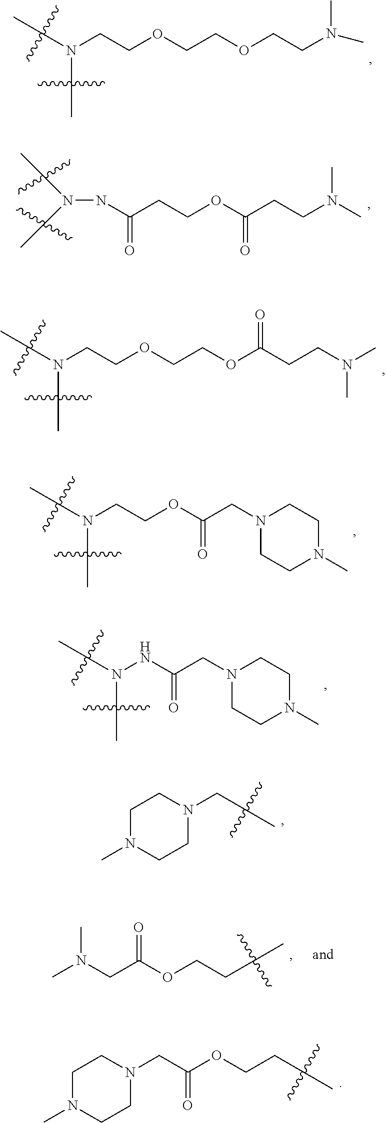

63. The multivesicular lipid nanoparticle composition of claim 56, wherein the carrier micelle and/or the sub-chamber inverse micelle each independently include a lipid having a formula described by:

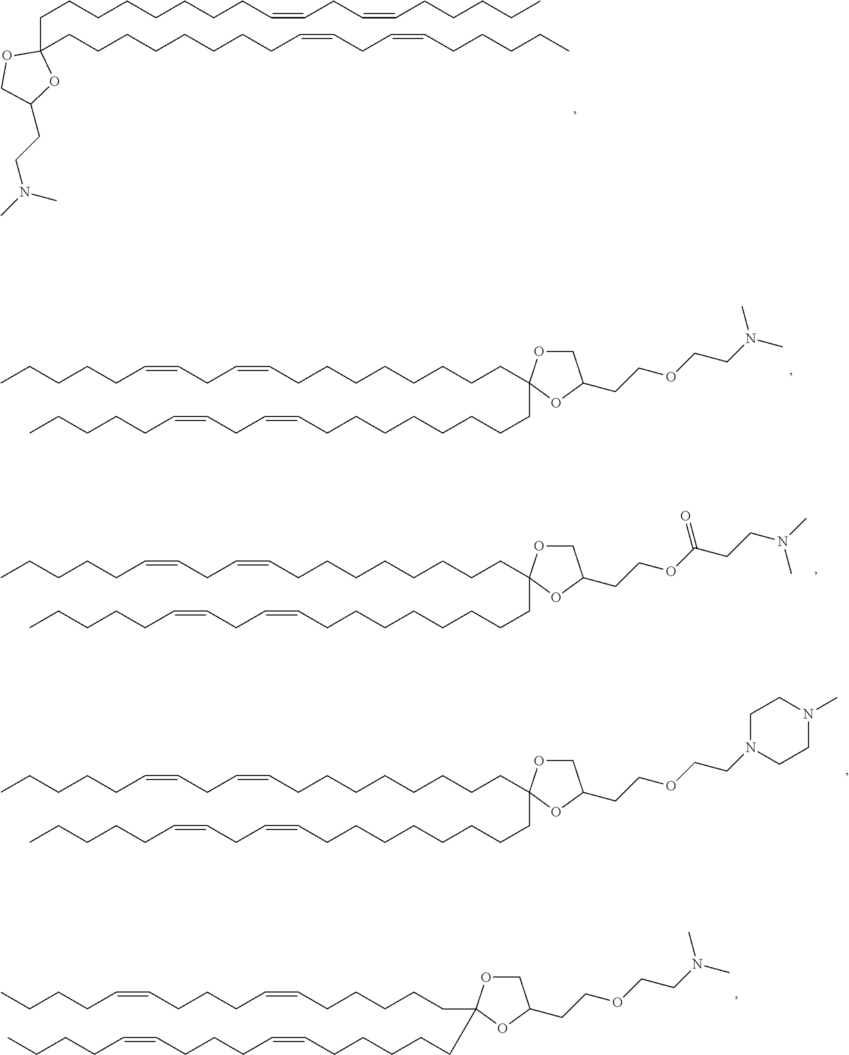

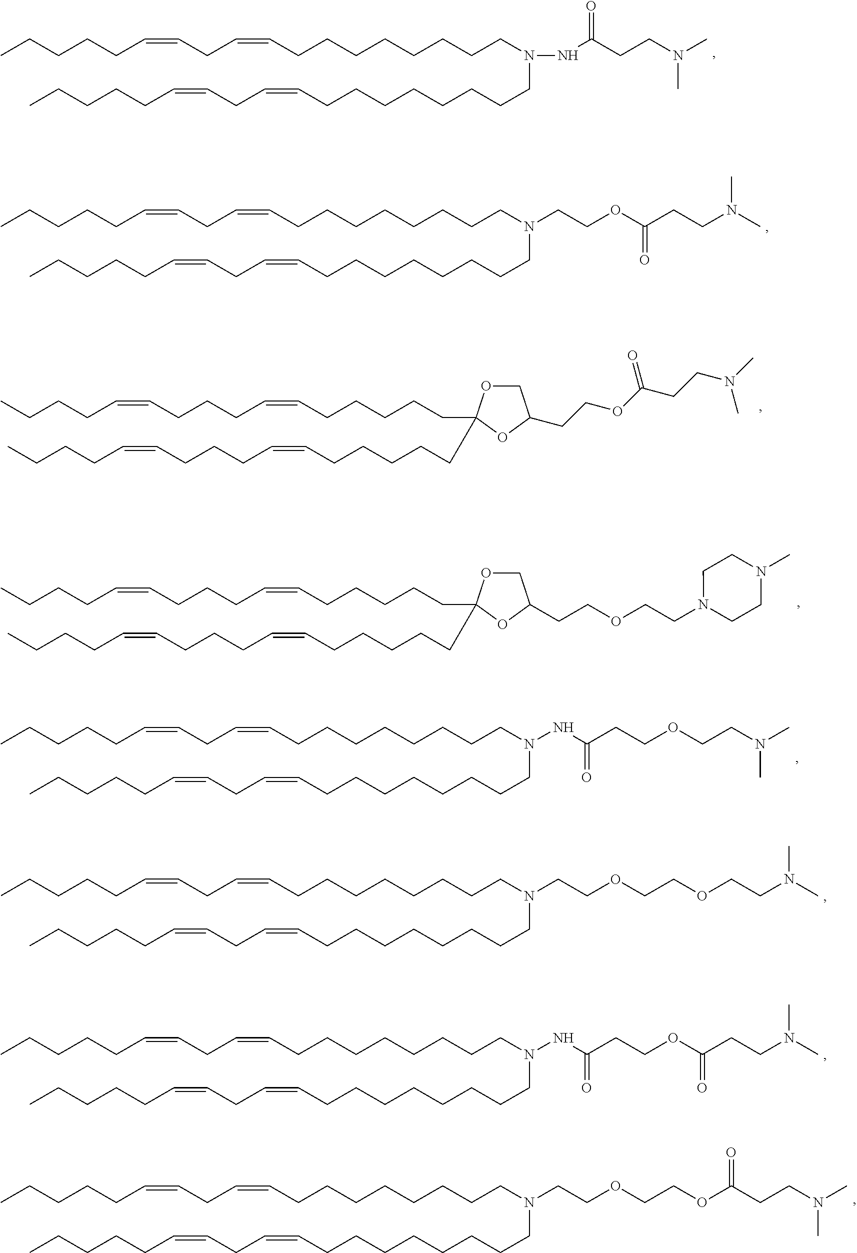

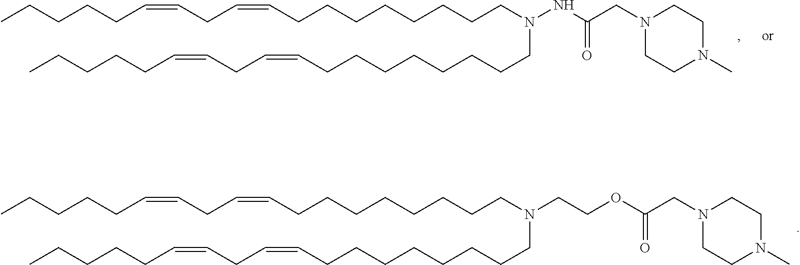

64. The multivesicular lipid nanoparticle composition of claim 56, wherein the carrier micelle and the sub-chamber inverse micelle each independently include conjugated tail group covalent linkages.

65. The multivesicular lipid nanoparticle composition of claim 56, wherein the sub-chamber inverse micelle includes 1,2-dipalmitoleoyl-sn-glycero-3-phosphocholine or residues thereof; 1,2-dipalmitoyl-sn-glycero-3-[phospho-rac-(3-lysyl(1-glycerol))](chloride salt) or residues thereof, 1-palmitoyl-2-[16-(acryloyloxy)palmitoyl]-sn-glycero-3-phosphorylcholine or residues thereof; 1,2-dipalmitoyl-sn-glycero-3-phosphoethanolamine-N-[methoxy(polyethyleneglycol)-2000](ammonium salt) or residues thereof, 2,2-dilinoleyl-4-dimethylaminoethyl-[1,3]-dioxolane or residues thereof, or Cholest-5-en-30-ol or residues thereof.

66. The multivesicular lipid nanoparticle composition of claim 56 further comprising a therapeutic/diagnostic payload encapsulated by at least one sub-chamber phospholipid inverse micelle and/or the carrier micelle.

67. The multivesicular lipid nanoparticle composition of claim 66, wherein the therapeutic/diagnostic payload is selected from the group consisting of small molecule drugs, polynucleotides, proteins, peptides, ribonucleoproteins, and combinations thereof.

68. The multivesicular lipid nanoparticle composition of claim 66, wherein the therapeutic/diagnostic payload includes a therapeutic immune tolerance induction payload selected from the group consisting of immune tolerance induction proteins, peptides, cytokines, polynucleotides, small molecules, and combinations thereof.

69. The multivesicular lipid nanoparticle composition of claim 66, wherein the therapeutic/diagnostic payload is a therapeutic payload selected from the group consisting of autoantigen proteins, autoantigen peptides, polynucleotides encoding autoantigen proteins, polynucleotides encoding autoantigen peptides, and combinations thereof.

70. The multivesicular lipid nanoparticle composition of claim 56, wherein conjugated tail group covalent linkages attach sub-chamber inverse micelle lipid nanoparticles to an inner surface of the carrier lipid nanoparticle.

71. The multivesicular lipid nanoparticle composition of claim 56, wherein the carrier micelle and the sub-chamber inverse micelle are crosslinked to each other.
